(19) United States
(12) Patent Application Publication
(10) Pub. No.: US 2021/0317111 A1
(43) Pub. Date: Oct. 14, 2021

(54) NOVEL MEDICAMENT FOR TREATING INFLAMMATORY BOWEL DISEASE

(71) Applicant: OTSUKA PHARMACEUTICAL CO., LTD., Tokyo (JP)

(72) Inventors: Isao SHIBUYA, Osaka-shi, Osaka (JP); Daisuke OKA, Osaka-shi, Osaka (JP); Kazuyuki FUJII, Osaka-shi, Osaka (JP); Hiroko TAKAGI, Osaka-shi, Osaka (JP); Masayoshi SATO, Osaka-shi, Osaka (JP); Takako NAKASHIMA, Osaka-shi, Osaka (JP); Fusako IWATA, Osaka-shi, Osaka (JP); Makoto MATSUMOTO, Osaka-shi, Osaka (JP)

(21) Appl. No.: 17/267,636

(22) PCT Filed: Aug. 9, 2019
(86) PCT No.: PCT/JP2019/031753

§ 371 Date: Feb. 10, 2021

(30) Foreign Application Priority Data
Aug. 13, 2018 (JP) 2018-152424

(51) Int. Cl. C07D 413/04; C07D 401/04; C07D 471/04; A61P 31/04; A61K 9/00

(52) U.S. Cl. CPC C07D 413/04; C07D 401/04; A61K 9/0053; A61P 31/04; C07D 471/04

(57) ABSTRACT

The present invention relates to a medicament for treating and/or preventing inflammatory bowel disease, comprising a quinolone compound of the formula shown below as an active ingredient.

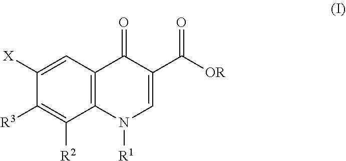

### TECHNICAL FIELD

The present invention relates to a medicament for treating and/or preventing inflammatory bowel disease, in more detail, a medicament for treating and/or preventing inflammatory bowel disease, comprising a quinolone compound as an active ingredient.

### BACKGROUND ART

Inflammatory bowel disease is a collective term of chronic diseases causing inflammation mainly in gastrointestinal tract, including Crohn's disease and ulcerative colitis, both of which are designated as incurable disease. Crohn's disease is an inflammatory disease that causes inflammation or ulcer in any regions of the whole gastrointestinal tract, from oral cavity to anus. In particular, Crohn's disease causes chronic inflammation or ulcer at mucosa of large intestine and small intestine. Ulcerative colitis is an inflammatory disease that occurs only in large intestine, which is different from Crohn's disease. The symptoms caused by these diseases includes abdominal pain, diarrhea, hematochezia, and weight loss, and the disease pathogenesis is complicated, which is thought to be various factors such as dietary habit, inheritance, enteric bacteria, and stress.

The current therapy of these inflammatory bowel diseases is carried out mainly with anti-inflammatory agents or immunosuppressive agents (Non-Patent Literatures 1 and 2), but the therapy has problems, i.e., insufficient effect, severe side effect, attenuation of the effect, risk of infection, etc. If the above agents show little therapeutic effects, another approach with antibiotics may be tried, but the use of antibiotics is limited because antibiotics may cause side effects and their effects are often not enough. Antibiotics for treating Crohn's disease which have been developed now include rifaximin, and a cocktail drug, RHB-104. Rifaximin has an action mechanism to decrease enteric bacteria which are related with the development of colitis (Non-Patent Literature 3). RHB-104 has an action mechanism based on antibacterial effect for Mycobacterium avium subspecies paratuberculosis which has been reported as pathogenic bacteria of Crohn's disease (Non-Patent Literature 4).

Patent Literature 1 discloses specific quinolone antibacterial agents which show the antibacterial activity for Clostridium difficile that occurs in intestinal tract.

### CITATION LIST

### Patent Literature

[PL 1] WO2013/029548

### Non-Patent Literature

[NPL 1] Journal of Autoimmunity 85 (2017) 103-116
[NPL 2] Gut June 2012 Vol 61, 918-932
[NPL 3] World J Gastroenterol 2011 Nov. 14; 17(42): 4643-4646
[NPL 4] Alcedo et al. Gut Pathog (2016) 8:32

### SUMMARY OF INVENTION

### Technical Problem

As mentioned above, although inflammatory bowel disease such as Crohn's disease causes severe inflammation in gastrointestinal tract, any effective therapeutic agents for leaving remission maintenance therapy have not been available until now. Thus, it has been desired to develop a new effective drug, in particular, a new drug having a different action mechanism from the already-existing drugs.

### Solution to Problem

The present inventors have found that a quinolone compound which is known to show the antibacterial activity for Clostridium difficile (hereinafter, also referred as to “the present compound”, including a pharmaceutically acceptable salt thereof) can unexpectedly show a potent effect for treating and preventing inflammatory bowel disease. The present inventors have further studied and then have found that the present compound has a potent antibacterial activity for bacteria which are related to inflammatory bowel disease, and additionally has unexpectedly a potent antiinflammatory, an inhibitory action for inflammatory cytokine (such as TNF-α) production, and an inhibitory action for the activation of T cell. Based upon the new findings, the present invention has been completed.

The present invention includes the following embodiments.

(Item 1)

A medicament comprising a quinolone compound of formula (I):

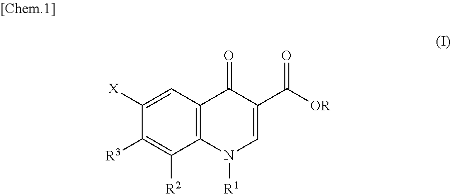

or a pharmaceutically acceptable salt thereof,
wherein
X is hydrogen atom or fluorine atom;
R is hydrogen atom or alkyl;
R¹ is (1) cyclopropyl which may be optionally substituted with 1 to 3 the same or different halogen atoms, or (2) phenyl which may be optionally substituted with 1 to 3 the same or different halogen atoms;
R² is hydrogen atom; alkyl which may be optionally substituted with 1 or 2 substituents selected independently from the group consisting of halogen atom and hydroxy; alkoxy; haloalkoxy; halogen atom; cyano; cyclopropyl; nitro; amino; formyl; alkenyl; or alkynyl; or
R¹ and R² are taken together to form 5- or 6-membered ring which may be optionally substituted with alkyl;

### R³ is

(1) a fused heterocyclyl group of the formula:

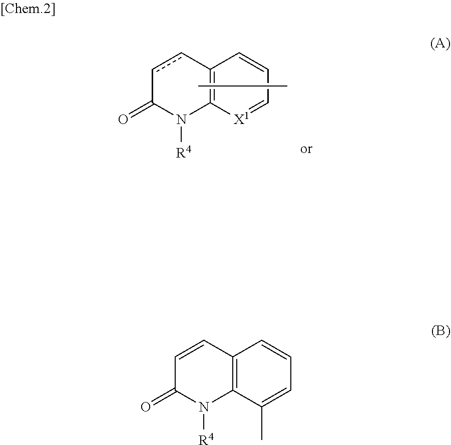

wherein

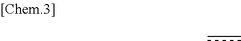

represents single bond or double bond,
X¹ is C(R⁵) or N,
R⁴ is hydrogen atom or alkyl,

### R⁵ is

(a) hydrogen atom,
(b) halogen atom,
(c) cyano,
(d) nitro,
(e) hydroxy,
(f) alkyl which may be optionally substituted with 1 to 3 the same or different halogen atoms,
(g) alkenyl or alkynyl,
(h) aryl, or
(i) alkoxy which may be optionally substituted with 1 to 3 the same or different halogen atoms,
when X¹ is C(R⁵), R⁴ and R⁵ may be taken together to form 5- or 6-membered ring which may be optionally substituted with oxo,
said fused heterocyclyl group may be optionally substituted with 1 or 2 substituents selected independently from the group consisting of halogen atom, cyano, nitro, hydroxy, and alkyl,
(2) a group of the formula:

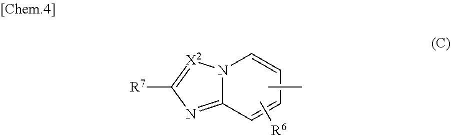

wherein
X² is C(R⁸) or N, and
R⁶, R⁷, and R⁸ are each independently,
(a) hydrogen atom,
(b) halogen atom,
(c) cyano,
(d) nitro,
(e) amino,
(f) alkyl which may be optionally substituted with 1 to 3 substituents selected independently from the group consisting of halogen atoms, alkoxy, and amino,
(g) alkenyl,
(h) alkynyl,
(i) aryl,
(j) formyl or CH═N—OH,
(k) carboxy,
(l) carbamoyl,
(m) 5- to 10-membered aromatic heterocyclyl group which may be optionally substituted with alkyl, or
(n) alkenyloxy,
(3) a group of the formula:

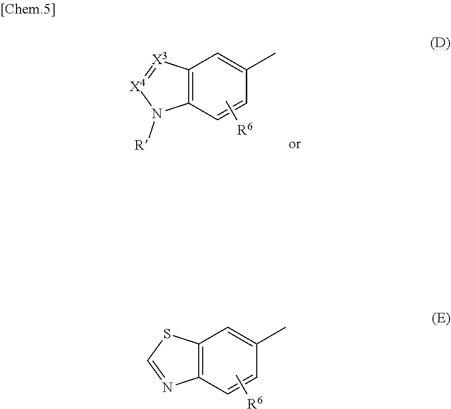

wherein
X³ and X⁴ are N, or
X³ is N, and X⁴ is CR″, wherein R″ is hydrogen atom; amino; hydroxy; alkyl which may be optionally substituted with 1 to 3 substituents selected independently from the group consisting of alkoxy and dimethylamino; or mercapto, or
X³ is CH and X⁴ is N,
R′ is hydrogen atom, or alkyl which may be optionally substituted with 1 to 3 substituents selected from the group consisting of substituted hydroxyl and amino, and
R⁶ is as defined above,
(4) a group of the formula:

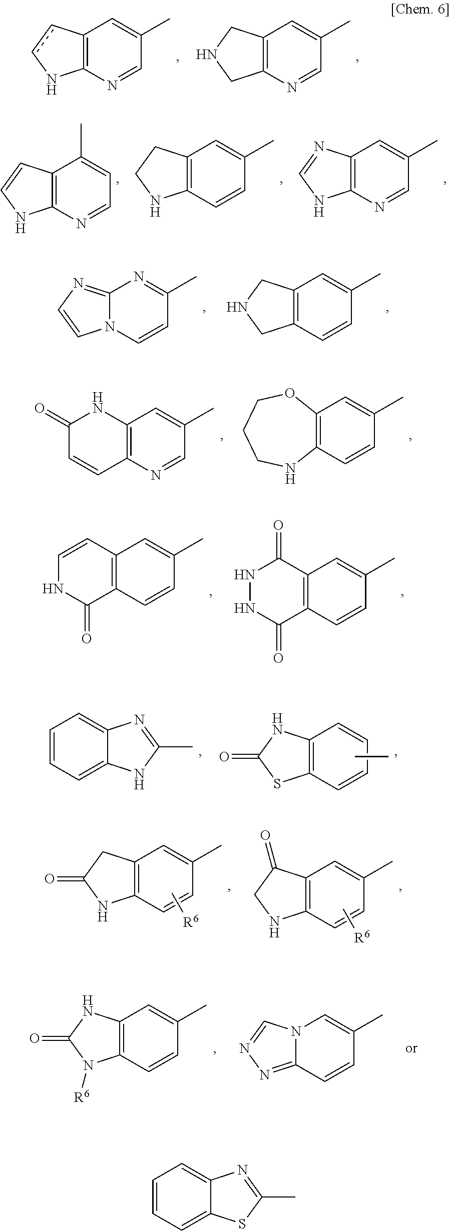

wherein

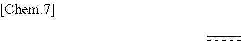

represents single bond or double bond, and R⁶ is as defined above,
(5) 3-pyridyl which may be optionally substituted with 1 to 2 substituents selected independently from the group consisting of the following (a)-(q):
(a) halogen atom,
(b) cyano,
(c) nitro,
(d) hydroxy,
(e) amino,
(f) alkyl which may be optionally substituted with 1 to 3 substituents selected independently from the group consisting of halogen atom, alkylamino, dialkylamino, and hydroxy,
(g) alkenyl or alkynyl,
(h) aryl,
(i) cycloalkyl,
(j) alkoxy,
(k) alkylamino,
(l) dialkylamino,
(m) phenylamino which may be optionally substituted with 1 to 3 the same or different halogen atoms,
(n) cyclic amino group which may be optionally substituted with alkoxycarbonyl,
(o) formyl,
(p) carbamoyl which may be optionally substituted with alkyl optionally-substituted with hydroxy, and
(q) 5- to 10-membered aromatic heterocyclyl group which may be optionally substituted with alkyl,
(6) 4-pyridyl which may be optionally substituted with halogen atom,
(7) 5-pyrimidinyl which may be optionally substituted with 1 or 2 substituents selected independently from the group consisting of amino, alkylamino, dialkylamino, and carboxy,
(8) 2-indolyl, 3-indolyl, 5-indolyl, 6-indolyl, benzofuranyl, benzothiophenyl, benzoxazolyl, or benzothiazolyl, each of which may be optionally substituted with 1 or 2 substituents selected independently from the consisting of the following (a)-(j):
(a) halogen atom,
(b) cyano,
(c) nitro,
(d) hydroxy,
(e) alkyl which may be optionally substituted with 1 to 3 substituents selected independently from the group consisting of amino, alkoxycarbonylamino, alkylamino, and dialkylamino,
(f) alkoxy,
(g) formyl,
(h) carboxy, and
(j) amino which may be optionally substituted with 1 or 2 substituents selected independently from the group consisting of the following (i)-(x):
(i) alkoxycarbonyl,
(ii) alkylcarbonyl which may be optionally substituted with a substituent selected from the group consisting of the following (A)-(E):
(A) cycloalkyloxy which may be optionally substituted with 1 to 3 the same or different alkyl,
(B) alkylamino,
(C) dialkylamino,
(D) cyclic amino group which may be optionally substituted with alkoxycarbonyl, and
(E) halogen atom,
(iii) phenylcarbonyl which may be optionally substituted with 1 to 3 substituents selected independently from the group consisting of alkyl and alkoxy,
(iv) cycloalkylcarbonyl,
(v) 5- to 10-membered aromatic heterocyclylcarbonyl group which may be optionally substituted with alkyl optionally-substituted with 1 to 3 the same or different halogen atoms,
(vi) benzylcarbonyl which may be substituted with 1 to 3 substituents selected independently from the group consisting of halogen atom and alkoxy,
(vii) arylsulfonyl which may be optionally substituted with alkoxy,
(viii) cycloalkylalkylsulfonyl which may be optionally substituted with 1 to 3 substituents selected independently from the group consisting of alkyl and oxo,
(ix) 5- to 10-membered aromatic heterocyclylsulfonyl group which may be optionally substituted with 1 to 3 the same or different alkyl, and
(x) —C(═N—CN)—SR⁹ wherein R⁹ is alkyl,
(9) a group of the formula:

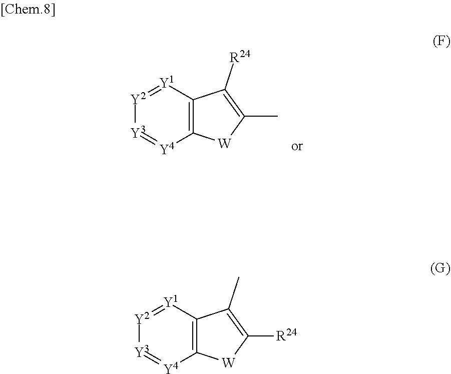

wherein
one of Y¹, Y², Y³, and Y⁴ is N or N⁺(—O⁻), and the remaining three are either different one of C(R²⁵), C(R²⁶), and C(R²⁷),

### W is O, S, or N(R²³),

R²³ is hydrogen atom or alkyl, and
R²⁴, R²⁵, R²⁶, and R²⁷ are each independently
(a) hydrogen atom,
(b) cyano, or
(c) nitro,
(10) a group of the formula:

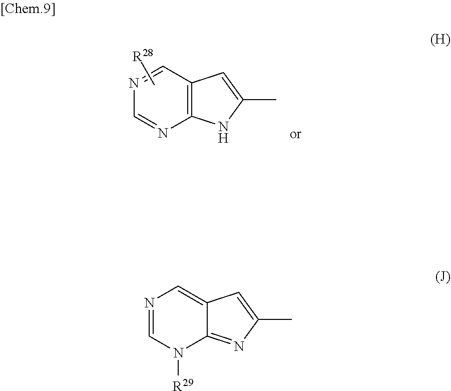

wherein
R²⁸ is hydrogen atom or hydroxy, and
R²⁹ is hydrogen atom or alkyl,
(11) a group of the formula:

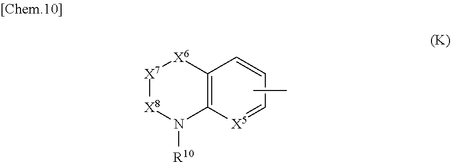

wherein
X⁵ is C(R¹¹) or N,
X⁶ is CH₂, C(═O), O, S, SO₂, or N(R¹²),
X⁷ is CH(R¹³), C(═O), or N(R¹⁴),
X⁸ is CH(R¹⁵) or C(═O),
R¹⁰, R¹², and R¹⁴ are each independently
(a) hydrogen atom or
(b) alkyl,
R¹¹, R¹³, and R¹⁵ are each independently
(a) hydrogen atom,
(b) halogen atom,
(c) cyano,
(d) nitro,
(e) amino,
(f) alkylamino,
(g) dialkylamino,
(h) alkyl which may be optionally substituted with hydroxy, or
(i) alkenyl,
when X⁵ is C(R¹¹), R¹⁰ and R¹¹ may be taken together to form 5- or 6-membered ring which may be optionally substituted with alkyl or oxo, and
when X⁶ is N(R¹²) and X⁷ is CH(R¹³), R¹² and R¹³ may be taken together to form 5- or 6-membered ring,
(12) a group of the formula:

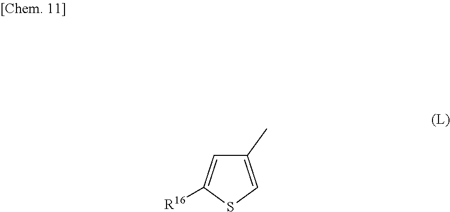

wherein R¹⁶ is
(a) hydrogen atom,
(b) alkyl which may be optionally substituted with 1 to 3 substituents selected from the group consisting of cyano, alkylamino, and dialkylamino,
(c) alkenyl which may be optionally substituted with carboxy,
(d) formyl,
(e) carboxy,
(f) carbamoyl,
(g) —C(R¹⁷)═N—OH wherein R¹⁷ is hydrogen atom, cyano, or hydroxy,
(h) 5- to 10-membered aromatic heterocyclyl group which may be optionally substituted with alkyl, alkoxycarbonyl, carboxy, or phenyl, or
(i) cyano,
(13) a group of the formula:

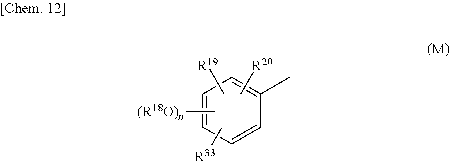

wherein
R¹⁸ is hydrogen atom, or alkyl which may be optionally substituted with 1 to 3 substituents selected independently from the group consisting of halogen atoms and phenyl,
n is 0 or 1,
R¹⁹, R²⁰, and R³³ are each independently,
(a) hydrogen atom,
(b) halogen atom,
(c) cyano,
(d) alkyl which may be optionally substituted with 1 to 3 substituents selected independently from the group consisting of the following (i)-(vii):
(i) halogen atom,
(ii) cyano,
(iii) hydroxy,
(iv) amino,
(v) alkylamino,
(vi) dialkylamino, and
(vii) cyclic amino group which may be optionally substituted with alkyl,
(e) alkoxy,
(f) amino which may be optionally substituted with 1 or 2 substituents selected independently from the following (i)-(v):
(i) alkylcarbonyl which may be optionally substituted with cyclic amino group,
(ii) alkylsulfonyl,
(iii) carbamoyl,
(iv) alkyl, cycloalkyl, or cycloalkylalkyl, and
(v) 5- to 10-membered saturated heterocyclyl,
(g) carboxy,
(h) alkoxycarbonyl,
(i) carbamoyl which may be optionally substituted with alkyl optionally-substituted with amino, alkylamino, dialkylamino, or alkoxycarbonylamino,
(j) formyl,
(k) 5- to 10-membered aromatic heterocyclyl group which may be optionally substituted with alkyl,
(l) —CH═N—OR²¹ wherein R²¹ is hydrogen atom, or alkyl which may be substituted with alkylamino or dialkylamino,
(m) nitro,
(n) 5- to 10-membered saturated heterocyclyl which may be optionally substituted with amino,
(o) phenyl, or

### (p) —NHC(SMe)=CHCN,

(14) a group of the formula:

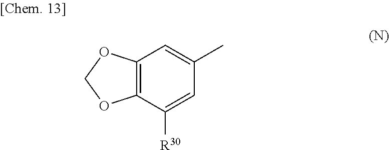

wherein

### R³⁰ is

(a) hydrogen atom,
(b) halogen atom,
(c) cyano,
(d) alkyl which may be optionally substituted with 1 to 3 substituents selected independently from the group consisting of halogen atom and hydroxy,
(e) alkenyl,
(f) alkynyl,
(g) alkoxy,
(h) formyl,

### (i) —CH═N—OH, or

(j) carbamoyl,
(15) naphthyl or isochromenyl,
(16) quinolyl or isoquinolyl, or oxide form thereof,
(17) a group of the formula:

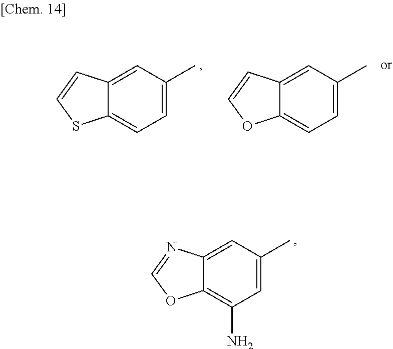

(18) a group of the formula:

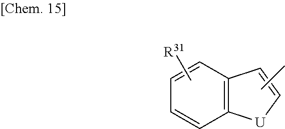

wherein

### U is O or S, and

### R³¹ is

(a) hydrogen atom,
(b) halogen atom,
(c) alkyl which may be optionally substituted with 1 to 3 the same or different halogen atoms,
(d) carboxy,
(e) nitro,
(f) cyano, or
(g) amino,
(19) a group of the formula:

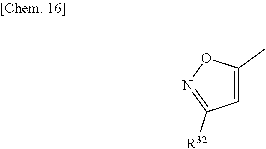

wherein

### R³² is

(a) halogen atom,
(b) phenyl, or
(c) a group of the formula:

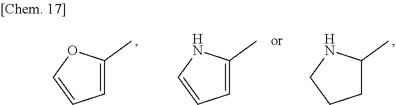

(20) a group of the formula:

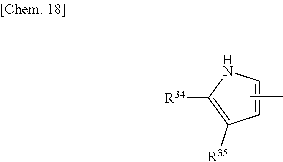

wherein
R³⁴ and R³⁵ are each independently
(a) hydrogen atom, or
(b) aminoalkyl, or
R³⁴ and R³⁵ are taken together to form 6-membered ring which may be optionally substituted with amino or oxo,
(21) a group of the formula:

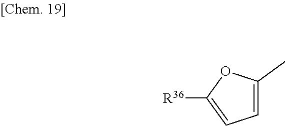

wherein R³⁶ is
(a) hydrogen atom,
(b) halogen atom,
(c) nitro, or
(d) thienyl, or
(22) a group of the formula:

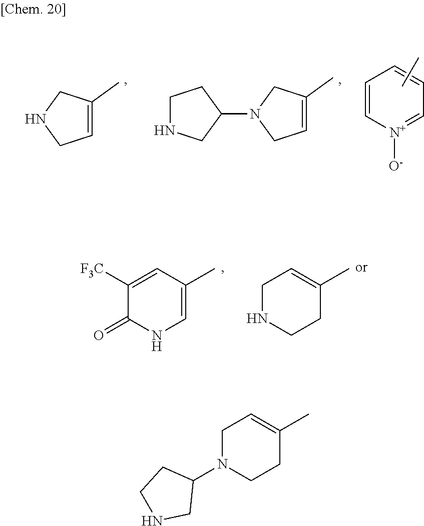

for treating and/or preventing a disease connected with the change of enteric bacteria, or a disease involving inflammation.

(Item 1′)

The medicament of Item 1, wherein the treatment and/or prevention is achieved by the antibacterial activity of the quinolone compound or a pharmaceutically acceptable salt thereof against enteric bacteria and the anti-inflammatory activity thereof.

(Item 2)

The medicament of Item 1 wherein the disease connected with the change of enteric bacteria, or the disease involving inflammation is inflammatory bowel disease.

(Item 3)

The medicament of Item 2 wherein the inflammatory bowel disease is Crohn's disease or ulcerative colitis.

(Item 4)

The medicament of Item 2 wherein the inflammatory bowel disease is Crohn's disease.

(Item 5)

The medicament of any one of Items 1-4, which is an oral preparation.

(Item 6)

The medicament of any one of Items 1-5, wherein the daily dose is 0.5 mg-6000 mg.

(Item 7)

A method for treating and/or preventing inflammatory bowel disease, comprising administering a therapeutically effective amount of the quinolone compound defined in Item 1 or a pharmaceutically acceptable salt thereof to a patient in need thereof.

(Item 8)

Use of the quinolone compound defined in Item 1 or a pharmaceutically acceptable salt thereof in the manufacture of a medicament for treating and/or preventing inflammatory bowel disease.

(Item 9)

The quinolone compound defined in Item 1 or a pharmaceutically acceptable salt thereof for use in treating and/or preventing inflammatory bowel disease.

(Item 10)

A medicament comprising the quinolone compound defined in Item 1 or a pharmaceutically acceptable salt thereof, which has an antibacterial activity against bacteria involved in inflammatory bowel disease, an inhibitory action for inflammatory cytokine production, and an inhibitory action for activation of T cell.

(Item 11)

The medicament of any one of Items 1 to 6, wherein X is fluorine atom.

(Item 12)

The medicament of any one of Items 1 to 6, wherein

R³ is a fused heterocyclyl group of the formula:

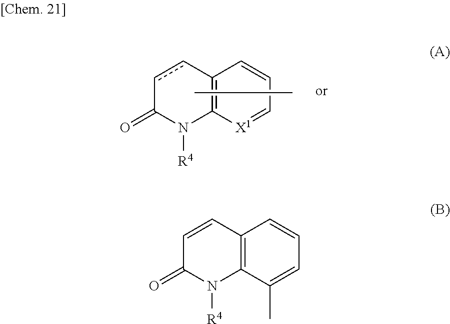

wherein

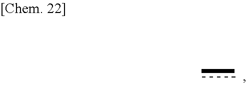

X¹, and R⁴ are as defined in Item 1, and said fused heterocyclyl group may be optionally substituted with 1 or 2 substituents selected independently from the group consisting of halogen atom, cyano, nitro, hydroxy, and alkyl.

(Item 13)

The medicament of any one of Items 1 to 6, wherein

R³ is a group of the formula:

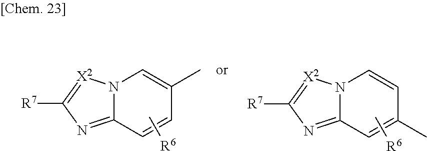

wherein X², R⁶, and R⁷ are as defined in Item 1.

(Item 14)

The medicament of any one of Items 1 to 6, wherein

R³ is a group of the formula:

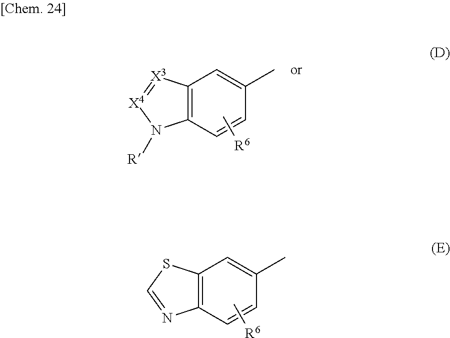

wherein X¹, X⁴, R⁶, and R′ are as defined in Item 1.

(Item 15)

The medicament of any one of Items 1 to 6, wherein

R³ is a group of the formula:

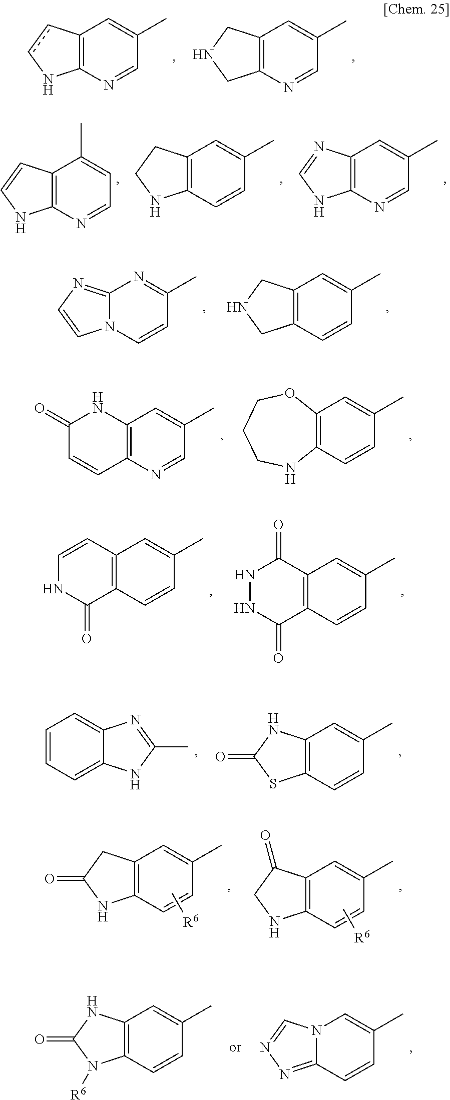

wherein

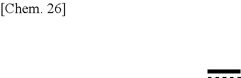

and R⁶ are as defined in Item 1.

(Item 16)

The medicament of any one of Items 1 to 6, wherein

R³ is a group of the formula:

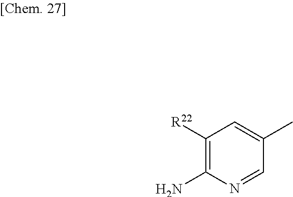

wherein R²² is
(a) halogen atom,
(b) cyano,
(c) nitro,
(d) alkyl which may be optionally substituted with 1 to 3 substituents selected independently from the group consisting of halogen atom, alkylamino, dialkylamino, and hydroxy,
(e) alkenyl or alkynyl,
(f) aryl,
(g) cycloalkyl,
(h) alkoxy,
(i) formyl, or
(j) carbamoyl which may be optionally substituted with alkyl optionally-substituted with hydroxyl.

(Item 17)

The medicament of any one of Items 1 to 6, wherein R³ is 5-pyrimidinyl substituted with 1 or 2 substituents selected independently from the group consisting of amino, alkylamino, dialkylamino, and carboxy.

(Item 18)

The medicament of any one of Items 1 to 6, wherein R³ is 2-indolyl which may be optionally substituted with 1 or 2 substituents selected independently from the group consisting of the following (a)-(j):

(a) halogen atom,

(b) cyano,

(c) nitro,

(d) hydroxy,

(e) alkyl which may be optionally substituted with 1 to 3 substituents selected independently from the group consisting of amino, alkoxycarbonylamino, alkylamino, and dialkylamino,

(f) alkoxy,

(g) formyl,

(h) carboxy, and

(j) amino which may be optionally substituted with 1 or 2 substituents selected independently from the group consisting of the following (i)-(x):

(i) alkoxycarbonyl,

(ii) alkylcarbonyl which may be optionally substituted with a substituent selected independently from the group consisting of the following (A)-(E):

(A) cycloalkyloxy which may be substituted with 1 to 3 the same or different alkyl,

(B) alkylamino,

(C) dialkylamino,

(D) cyclic amino group which may be optionally substituted with alkoxycarbonyl, and

(E) halogen atom,

(iii) phenylcarbonyl which may be optionally substituted with 1 to 3 substituents selected independently from the group consisting of alkyl and alkoxy,

(iv) cycloalkylcarbonyl,

(v) 5- to 10-membered aromatic heterocyclylcarbonyl group which may be optionally substituted with alkyl optionally-substituted with 1 to 3 the same or different halogen atoms,

(vi) benzylcarbonyl which may be optionally substituted with 1 to 3 substituents selected independently from the group consisting of halogen atom and alkoxy,

(vii) arylsulfonyl which may be optionally substituted with alkoxy,

(viii) cycloalkylalkylsulfonyl which may be optionally substituted with 1 to 3 substituents selected independently from the group consisting of alkyl and oxo,

(ix) 5- to 10-membered aromatic heterocyclylsulfonyl group which may be optionally substituted with 1 to 3 the same or different alkyl, and

(x) —C(═N—CN)—SR⁹ wherein R⁹ is alkyl.

(Item 19)

The medicament of any one of Items 1 to 6, wherein R³ is a group of the formula:

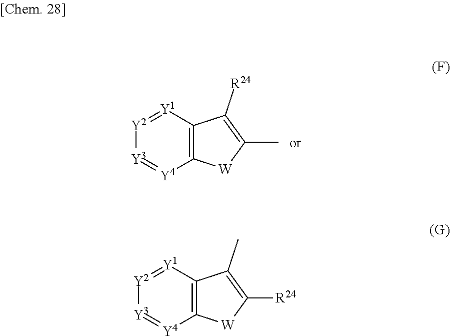

wherein Y¹, Y², Y³, Y⁴, W, and R²⁴ are as defined in Item 1.

(Item 20)

The medicament of any one of Items 1 to 6, wherein R³ is a group of the formula:

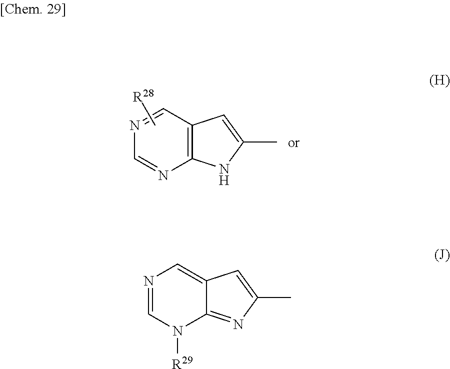

wherein R²⁸ and R²⁹ are as defined in Item 1.

(Item 21)

The medicament of any one of Items 1 to 6, wherein R³ is a group of the formula:

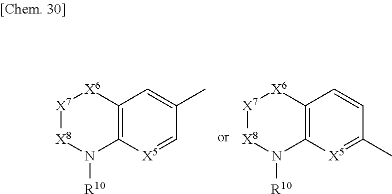

wherein X⁵, X⁶, X⁷, X⁸, and R¹⁰ are as defined in Item 1.

(Item 22)

The medicament of any one of Items 1 to 6, wherein R³ is a group of the formula:

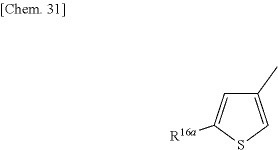

wherein R^{16a} is

(a) alkyl which may be optionally substituted with 1 to 3 substituents selected independently from the group consisting of cyano, alkylamino, and dialkylamino,

(b) alkenyl which may be optionally substituted with carboxy,

(c) formyl,

(d) carboxy,

(e) carbamoyl,

(f) —C(R¹⁷)═N—OH wherein R¹⁷ is hydrogen atom, cyano, or hydroxy,

(g) 5- to 10-membered aromatic heterocyclyl group which may be optionally substituted with alkyl, alkoxycarbonyl, carboxy, or phenyl, or

(h) cyano.

(Item 23)

The medicament of any one of Items 1 to 6, wherein R³ is a group of the formula:

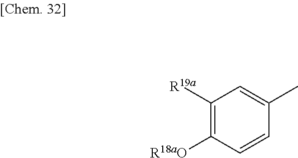

wherein

R^{18a} is alkyl,

R^{19a} is

(a) halogen atom,

(b) cyano,

(c) alkyl which may be optionally substituted with 1 to 3 substituents selected independently from the group consisting of the following (i)-(vii):

(i) halogen atom,

(ii) cyano,

(iii) hydroxy,

(iv) amino,

(v) alkylamino,

(vi) dialkylamino, and

(vii) cyclic amino group which may be optionally substituted with alkyl,

(d) alkoxy,

(e) amino which may be optionally substituted with 1 or 2 substituents selected independently from the group consisting of the following (i)-(iv):

(i) alkylcarbonyl which may be optionally substituted with cyclic amino group,

(ii) alkylsulfonyl,

(iii) carbamoyl, and

(iv) alkyl or cycloalkyl,

(f) carboxy,

(g) alkoxycarbonyl,

(h) carbamoyl which may be optionally substituted with alkyl optionally-substituted with amino, alkylamino, dialkylamino, or alkoxycarbonylamino,

(i) formyl,

(j) 5- to 10-membered aromatic heterocyclyl group which may be optionally substituted with alkyl,

(k) —CH═N—OR²¹ wherein R²¹ is hydrogen atom, or alkyl which may be optionally substituted with alkylamino or dialkylamino, or

(l) nitro.

(Item 24)

The medicament of any one of Items 1 to 6, wherein R³ is a group of the formula:

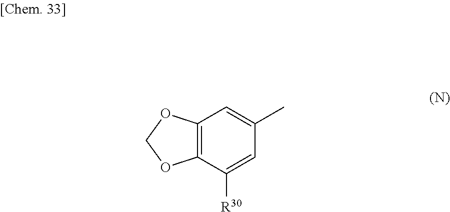

wherein R³⁰ is as defined in Item 1.

(Item 25)

The medicament of any one of Items 1 to 6, wherein R³ is naphthyl or isochromenyl.

(Item 26)

The medicament of any one of Items 1 to 6, wherein R³ is quinolyl or isoquinolyl.

(Item 27)

The medicament of any one of Items 1 to 6, wherein R is hydrogen atom.

(Item 28)

The medicament of any one of Items 1 to 6, wherein R¹ is cyclopropyl, 2-fluorocyclopropyl, or 2,4-difluorophenyl.

(Item 29)

The medicament of any one of Items 1 to 6, wherein R² is methyl, methoxy or chlorine atom.

(Item 30)

The medicament of Item 1, wherein the quinolone compound is selected from the group consisting of formula:

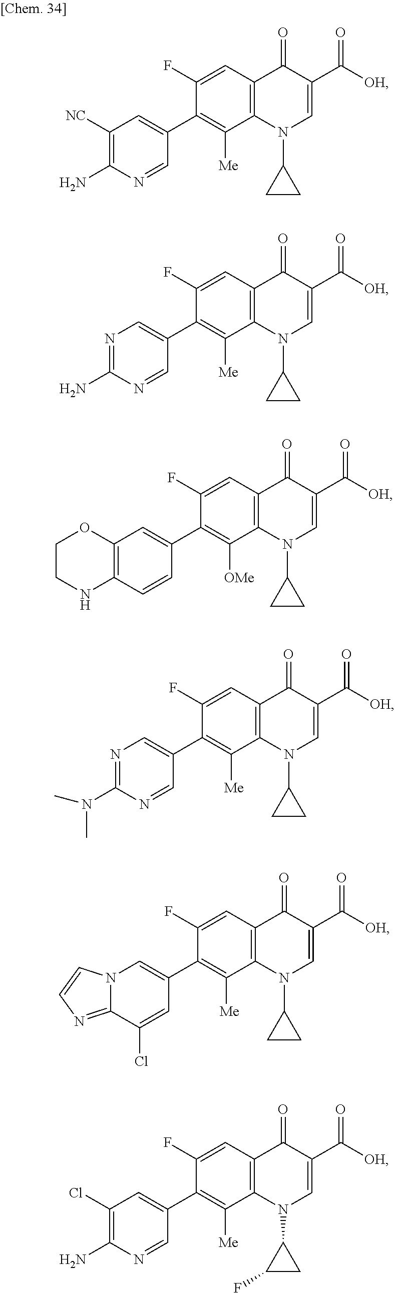

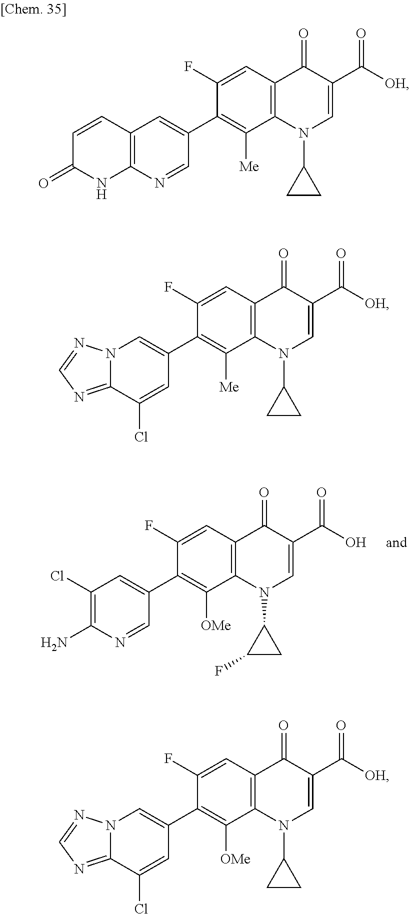 [SMILES: Cc1c(-c2cnc3[nH]c(=O)ccc3c2)c(F)cc2c(=O)c(C(=O)O)cn(C3CC3)c12 ; Cc1c(-c2cc(Cl)c3ncnn3c2)c(F)cc2c(=O)c(C(=O)O)cn(C3CC3)c12 ; COc1c(-c2cnc(N)c(Cl)c2)c(F)cc2c(=O)c(C(=O)O)cn([C@@H]3C[C@@H]3F)c12 ; "and" ; COc1c(-c2cc(Cl)c3ncnn3c2)c(F)cc2c(=O)c(C(=O)O)cn(C3CC3)c12]

or a salt thereof.

The medicament of Item 1, wherein the quinolone compound is selected from the following compounds or a salt thereof:
7-(6-amino-5-cyanopyridin-3-yl)-1-cyclopropyl-6-fluoro-1,4-dihydro-8-methyl-4-oxo-3-quinoline-carboxylic acid,
7-(2-amino-pyrimidin-5-yl)-1-cyclopropyl-6-fluoro-1,4-dihydro-8-methyl-4-oxo-3-quinoline-carboxylic acid,
7-(3,4-dihydro-2H-benzo[b][1,4]oxazin-7-yl)-1-cyclopropyl-6-fluoro-1,4-dihydro-8-methoxy-4-oxo-3-quinoline-carboxylic acid,
7-(2-dimethylamino-pyrimidin-5-yl)-1-cyclopropyl-6-fluoro-1,4-dihydro-8-methyl-4-oxo-3-quinoline-carboxylic acid,
7-(8-chloroimidazo[1,2-a]pyridin-6-yl)-1-cyclopropyl-6-fluoro-1,4-dihydro-8-methyl-4-oxo-quinoline-3-carboxylic acid,
7-(6-amino-5-chloropyridin-3-yl)-1-((1R,2S)-2-fluorocyclopropyl)-6-fluoro-1,4-dihydro-8-methyl-4-oxo-quinoline-3-carboxylic acid,
7-(7-oxo-7,8-dihydro-1,8-naphthyridin-3-yl)-1-cyclopropyl-6-fluoro-1,4-dihydro-8-methyl-4-oxo-quinoline-3-carboxylic acid,
7-(8-chloro-[1,2,4]triazolo[1,5-a]pyridin-6-yl)-1-cyclopropyl-6-fluoro-1,4-dihydro-8-methyl-4-oxo-quinoline-3-carboxylic acid,
7-(6-amino-5-chloropyridin-3-yl)-1-((1R,2S)-2-fluorocyclopropyl)-6-fluoro-1,4-dihydro-8-methoxy-4-oxo-quinoline-3-carboxylic acid, and
7-(8-chloro-[1,2,4]triazolo[1,5-a]pyridin-6-yl)-1-cyclopropyl-6-fluoro-1,4-dihydro-8-methoxy-4-oxo-quinoline-3-carboxylic acid.

### Effect of Invention

According to the present invention, it is thought that the present compound has an effect treating and/or preventing for a disease connected with the change of enteric bacteria, or a disease involving inflammation by its antibacterial activity against enteric bacteria and its anti-inflammatory activity. For example, said disease includes inflammatory bowel disease, more preferably Crohn's disease and ulcerative colitis. In particular, the present compound exhibited a high mucosal healing rate and showed improvement of stool symptom and the like, by short-term administration in gastrointestinal tract of an animal model of inflammatory bowel disease. Thus, the present invention is expected to be an excellent drug for treating inflammatory bowel disease, which can rapidly improve the symptom (i.e., rapidly remission induction) and can completely healing ulcer (i.e., complete cure). In addition, the present compound exhibits an inhibitory action for inflammatory cytokine (such as TNF-α) production and an inhibitory action for the activation of T cell, and exhibits an antibacterial activity effect against bacteria connected with inflammatory bowel disease whose effect is the same or more than that of the antibiotics which have been now developed for treating Crohn's disease. Thus, the present invention is expected to be an excellent drug for treating inflammatory bowel disease which has new action mechanism, i.e., having anti-inflammatory effect and antibacterial activity effect, for inflammatory bowel disease. Furthermore, the present compound is a poorly absorbable drug, and thereby it is distributed in a high concentration in the intestinal tract when it is orally administered, but it has low blood transferability. Thus, the present compound also has a merit, i.e., a low risk of generalized side effect, which is a problem in existing quinolone antibacterial agents.

### BRIEF DESCRIPTION OF DRAWINGS

FIG. 1 shows the histological score results of Test 1 in Example 1.

FIG. 2 shows the histological score results of Test 2 in Example 1.

FIG. 3 shows the histological score results of Test 3 in Example 1.

FIG. 4 shows the results of Example 2.

FIG. 5 shows the results of Example 3.

FIG. 6 shows the results of the rate of activated T cell in Example 4.

FIG. 7 shows the results of the cytokine production in Example 4.

FIG. 8 shows the results of the rate of proliferated T cell in Example 4.

FIG. 9 shows the results of the stool consistency score in Example 8. The stool consistency score when the present compound at a dose of 1, 3, 10 mg/kg or SASP at a dose of 100 mg/kg was orally administered twice daily in DSS-induced colitis (mean±standard error).

FIG. 10 shows the results of the bloody stool score in Example 8. The bloody stool score when the present compound at a dose of 1, 3, 10 mg/kg or SASP at a dose of 100 mg/kg was orally administered twice daily in DSS-induced colitis (mean±standard error).

FIG. 11 shows the results of the total stool score in Example 8. The total stool score when the present compound at a dose of 1, 3, 10 mg/kg or SASP at a dose of 100 mg/kg was orally administered twice daily in DSS-induced colitis (mean±standard error).

### DESCRIPTION OF EMBODIMENTS

Specific examples of each group in the compound of formula (I) may be shown as follows.

The “halogen atom” includes fluorine atom, chlorine atom, bromine atom, and iodine atom.

The “alkyl” and the “alkyl” moiety in “alkylamino”, “dialkylamino”, “alkylcarbonyl”, “cycloalkylalkylsulfonyl”, “cycloalkylalkyl”, “aminoalkyl”, and “alkylsulfonyl” include straight or branched C₁₋₆ alkyl such as methyl, ethyl, propyl, isopropyl, butyl, isobutyl, sec-butyl, tert-butyl, pentyl, 1-ethylpropyl, isopentyl, neopentyl, tert-pentyl, hexyl, 1,2,2-trimethylpropyl, 3,3-dimethylbutyl, 2-ethylbutyl, and isohexyl, 3-methylpentyl.

The “alkenyl” includes straight or branched C₂₋₆ alkenyl such as vinyl, 1-propenyl, 2-propenyl, 1-butenyl, 2-butenyl, 3-butenyl, 1-methyl-2-propenyl, 2-pentenyl, and 2-hexenyl.

The “alkynyl” includes straight or branched C₂₋₆ alkynyl such as ethinyl, 2-propynyl, 2-butynyl, 3-butynyl, 1-methyl-2-propynyl, 2-pentynyl, and 2-hexynyl.

The “alkoxy” and the “alkoxy” moiety in “haloalkoxy”, “alkoxycarbonyl”, and “alkoxycarbonylamino” include straight or branched C₁₋₆ alkoxy such as methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, and 3-methylpentyloxy.

The “haloalkoxy” includes straight or branched C₁₋₆ alkoxy substituted with 1 to 3 the same or different halogen atoms, which includes, for example, fluoromethoxy, difluoromethoxy, trifluoromethoxy, chloromethoxy, dichloromethoxy, trichloromethoxy, bromomethoxy, dibromomethoxy, dichlorofluoromethoxy, 2,2,2-trifluoroethoxy, 2-chloroethoxy, 3,3,3-trifluoropropoxy, 2-chloropropoxy, 3-chloropropoxy, 3-bromopropoxy, 4,4,4-trifluorobutoxy, 2-chlorobutoxy, 4-chlorobutoxy, 4-bromobutoxy, 5,5,5-trifluoropentyloxy, 5-chloropentyloxy, 6,6,6-trifluorohexyloxy, and 6-chlorohexyloxy; preferably, difluoromethoxy.

The “alkenyloxy” includes straight or branched C₂₋₆ alkenyloxy such as vinyloxy, 1-propenyloxy, 2-propenyloxy, 1-butenyloxy, 2-butenyloxy, 3-butenyloxy, 1-methyl-2-propenyloxy, 2-pentenyloxy, and 2-hexenyloxy.

The “aryl” and the “aryl” moiety in “arylsulfonyl” include C₆₋₁₄ (preferably, C₆₋₁₀) aryl such as phenyl, and naphthyl (e.g. 1-naphthyl, 2-naphthyl). Preferably, it includes phenyl.

The “5- to 10-membered aromatic heterocyclyl group” and the “5- to 10-membered aromatic heterocyclyl” moiety in “5- to 10-membered aromatic heterocyclylcarbonyl group” and “5- to 10-membered aromatic heterocyclylsulfonyl group” include 5- to 10-membered (preferably, 5- or 6-membered) aromatic heterocyclyl group containing 1 to 4 (preferably, 1 to 3; more preferably, 1 or 2) heteroatoms selected independently from nitrogen atom, oxygen atom, and sulfur atom. It includes, for example, furyl, thienyl, pyrrolyl, pyrazolyl, imidazolyl, triazolyl (e.g. 1,2,3-triazolyl, 1,2,4-triazolyl), tetrazolyl, isoxazolyl, oxazolyl, furazanyl, isothiazolyl, thiazolyl, pyridyl (e.g. 2-pyridyl, 3-pyridyl, 4-pyridyl), pyridazinyl, pyrimidinyl, pyrazinyl, benzofuranyl, isobenzofuranyl, benzo[b]thiophenyl, benzo[c]thiophenyl, indolyl, isoindolyl, indolizinyl, indazolyl, benzimidazolyl, benzotriazolyl, benzoxazolyl, 1,2-benzisoxazolyl, benzothiazolyl, 1,2-benzisothiazolyl, purinyl, quinolyl, isoquinolyl, quinolizinyl, cinnolinyl, quinazolinyl, quinoxalinyl, phthalazinyl, naphthyridinyl, and pteridinyl; preferably, pyrrolyl, imidazolyl, oxazolyl, triazolyl (e.g. 1,2,3-triazolyl, 1,2,4-triazolyl), tetrazolyl, pyridyl (e.g. 2-pyridyl, 3-pyridyl, 4-pyridyl), and benzimidazolyl.

The “alkylamino” includes C₁₋₆ alkylamino such as methylamino, ethylamino, propylamino, isopropylamino, butylamino, isobutylamino, sec-butylamino, tertbutylamino, pentylamino, isopentylamino, neopentylamino, tert-pentylamino, and hexylamino.

The “dialkylamino” includes di(C₁₋₆ alkyl)amino such as dimethylamino, diethylamino, dipropylamino, diisopropylamino, dibutylamino, diisobutylamino, di(sec-butyl)amino, di(tert-butyl)amino, dipentylamino, di(tert-pentyl)amino, dihexylamino, and ethylmethylamino.

The “aminoalkyl” includes amino-C₁₋₆ alkyl such as aminomethyl, 2-aminoethyl, 3-aminopropyl, 4-aminobutyl, 5-aminopentyl, and 6-aminohexyl.

The “cycloalkyl” and the “cycloalkyl” moiety in “cycloalkyloxy”, “cycloalkylcarbonyl”, “cycloalkylalkyl”, and “cycloalkylalkylsulfonyl” include C₃₋₈ cycloalkyl such as cyclopropyl, cyclobutyl, cyclopentyl, cyclohexyl, cycloheptyl, cyclooctyl, and norbornanyl (e.g. 2-norbornanyl).

The “cycloalkylalkyl” includes C₃₋₈ cycloalkyl-C₁₋₆ alkyl such as cyclopropylmethyl, cyclobutylmethyl, cyclopentylmethyl, cyclohexylmethyl, cycloheptylmethyl, cyclooctylmethyl, and norbornanylmethyl (e.g. norbornan-2-ylmethyl).

The “cyclic amino group” includes 4- to 7-membered (preferably, 5- or 6-membered) cyclic amino group containing one nitrogen atom, and optionally further containing one heteroatom selected from nitrogen atom, oxygen atom, and sulfur atom. It includes, for example, 1-azetidinyl, 1-pyrrolidinyl, 1-imidazolidinyl, 1-pyrazolidinyl, piperidino, 1-piperazinyl, morpholino, thiomorpholino, 1-azepanyl, and 1,4-oxazepan-4-yl; preferably, 1-pyrrolidinyl, piperidino, 1-piperazinyl, morpholino, and thiomorpholino.

The “alkoxycarbonyl” includes C₁₋₆ alkoxy-carbonyl wherein the alkoxy moiety is C₁₋₆ alkoxy, which includes, for example, methoxycarbonyl, ethoxycarbonyl, propoxycarbonyl, isopropoxycarbonyl, butoxycarbonyl, isobutoxycarbonyl, secbutoxycarbonyl, tert-butoxycarbonyl, pentyloxycarbonyl, and hexyloxycarbonyl.

The “alkoxycarbonylamino” includes C₁₋₆ alkoxy-carbonylamino wherein the alkoxy moiety is C₁₋₆ alkoxy, which includes, for example, methoxycarbonylamino, ethoxycarbonylamino, propoxycarbonylamino, isopropoxycarbonylamino, butoxycarbonylamino, isobutoxycarbonylamino, sec-butoxycarbonylamino, tertbutoxycarbonylamino, pentyloxycarbonylamino, and hexyloxycarbonylamino.

The “alkylcarbonyl” includes C₁₋₆ alkyl-carbonyl wherein the alkyl moiety is C₁₋₆ alkyl, which includes, for example, acetyl, ethylcarbonyl, propylcarbonyl, isopropylcarbonyl, butylcarbonyl, isobutylcarbonyl, sec-butylcarbonyl, tert-butylcarbonyl, pentylcarbonyl, and hexylcarbonyl.

The “cycloalkyloxy” includes C₃₋₈ cycloalkyloxy such as cyclopropyloxy, cyclobutyloxy, cyclopentyloxy, cyclohexyloxy, cycloheptyloxy, and cyclooctyloxy.

The “cycloalkylcarbonyl” includes C₃₋₈ cycloalkyl-carbonyl such as cyclopropylcarbonyl, cyclobutylcarbonyl, cyclopentylcarbonyl, cyclohexylcarbonyl, cycloheptylcarbonyl, and cyclooctylcarbonyl.

The “5- to 10-membered aromatic heterocyclylcarbonyl group” includes 5- to 10-membered (preferably, 5- or 6-membered) aromatic heterocyclylcarbonyl group, wherein the heterocyclyl moiety contains 1 to 4 (preferably 1 to 3, more preferably 1 or 2) heteroatoms selected independently from nitrogen atom, oxygen atom, and sulfur atom. Examples of the heterocyclyl moiety are the same as the examples of the 5- to 10-membered aromatic heterocyclyl group mentioned above. Preferred examples of the “5- to 10-membered aromatic heterocyclylcarbonyl group” include pyridylcarbonyl (e.g. 2-pyridylcarbonyl, 3-pyridylcarbonyl, 4-pyridylcarbonyl).

The “arylsulfonyl” includes C₆₋₁₄ (preferably C₆₋₁₀) arylsulfonyl such as phenylsulfonyl and naphthylsulfonyl (e.g. 1-naphthylsulfonyl, 2-naphthylsulfonyl). Preferred example thereof includes phenylsulfonyl.

The “cycloalkylalkylsulfonyl” includes C₃₋₈ cycloalkyl-C₁₋₆ alkylsulfonyl such as cyclopropylmethylsulfonyl, cyclobutylmethylsulfonyl, cyclopentylmethylsulfonyl, cyclohexylmethylsulfonyl, cycloheptylmethylsulfonyl, cyclooctylmethylsulfonyl, and norbornanylmethylsulfonyl (e.g. norbornan-2-ylmethylsulfonyl).

The “5- to 10-membered aromatic heterocyclylsulfonyl group” includes 5- to 10-membered (preferably 5- or 6-membered) aromatic heterocyclylsulfonyl group, wherein the heterocyclyl moiety contains 1 to 4 (preferably 1 to 3, more preferably 1 or 2) heteroatoms selected independently from nitrogen atom, oxygen atom, and sulfur atom. Examples of the heterocyclyl moiety are the same as the examples of the 5- to 10-membered aromatic heterocyclyl group mentioned above. Preferred examples of the “5- to 10-membered aromatic heterocyclylsulfonyl group” includes imidazolylsulfonyl.

The “alkylsulfonyl” includes C₁₋₆ alkylsulfonyl wherein the alkyl moiety is C₁₋₆ alkyl. Examples thereof include methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, and hexylsulfonyl.

The “cyclopropyl which may be optionally substituted with 1 to 3 the same or different halogen atoms” includes cyclopropyl optionally-substituted with one fluorine atom such as cyclopropyl and 2-fluorocyclopropyl.

The “phenyl which may be optionally substituted with 1 to 3 the same or different halogen atoms” includes phenyl substituted with two fluorine atoms such as 2,4-difluorophenyl.

The “5- to 10-membered saturated heterocyclyl” includes 5- to 10-membered (preferably 5- or 6-membered) saturated heterocyclyl containing 1 to 4 (preferably 1 to 3, more preferably 1 or 2) heteroatoms selected independently from nitrogen atom, oxygen atom, and sulfur atom. Examples thereof include pyrrolidinyl, piperidyl, piperazinyl, morpholinyl, and thiomorpholinyl.

The “6-membered ring which may be optionally substituted with amino or oxo” formed by R³⁴ and R³⁵ includes 6-membered ring optionally-containing one nitrogen atom, said ring is optionally substituted with amino or oxo. Examples thereof include cyclohexene and dihydropyridine, which may be optionally substituted with amino or oxo.

The “5- or 6-membered ring which may be optionally substituted with alkyl” formed by R¹ and R² includes 5- or 6-membered (preferably 6-membered) ring containing one nitrogen atom and optionally further containing one oxygen atom, and said ring is optionally substituted with alkyl. Preferably, R¹ and R² may be optionally taken together to form —O—CH₂—CH(CH₃)— wherein the oxygen atom is bonded to the phenyl ring in the quinolone ring as shown below.

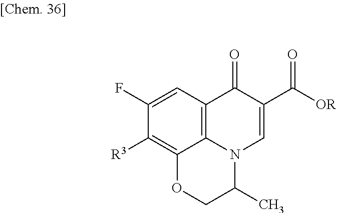

Wherein R³ is as defined above.

The “5- or 6-membered ring which may be optionally substituted with oxo” formed by R⁴ and R⁵ includes 5- or 6-membered (preferably 6-membered) ring containing one nitrogen atom and optionally further containing one oxygen atom, said ring is optionally substituted with oxo. Preferably, R⁴ and R⁵ may be optionally taken together to form —CH₂—O—(C═O)— wherein the carbonyl is bonded to the phenyl ring in the quinolone ring as shown below.

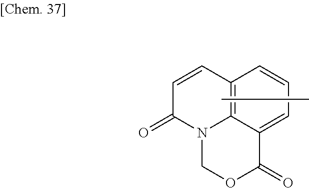

The “5- or 6-membered ring which may be optionally substituted with alkyl or oxo” formed by R¹⁰ and R¹¹ includes 5- or 6-membered (preferably 5-membered) ring containing 2 or 3 nitrogen atoms, said ring is optionally substituted with alkyl or oxo. Preferably, R¹⁰ and R¹¹ may be optionally taken together to form —(C═O)—NH—, —C(R³¹)═N—, or —N═N— wherein R³¹ is hydrogen atom or alkyl, and the nitrogen atom is bonded to the phenyl ring in the fused ring as shown below.

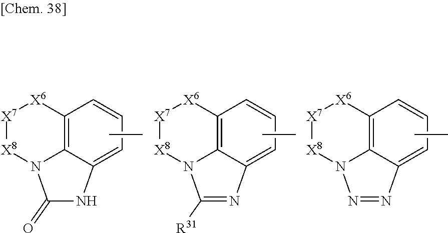

Wherein X⁶, X⁷, X⁸, and R³¹ are as defined above.

The “5- or 6-membered ring” formed by R¹² and R¹³ includes 5- or 6-membered (preferably 6-membered) ring containing one nitrogen atom. Preferably, R¹² and R¹³ may be optionally taken together to form —(CH₂)₄— as shown below.

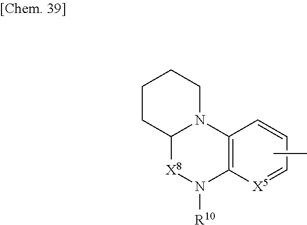

Wherein X⁵, X⁸, and R¹⁰ are as defined above.

For example, as shown in the formula:

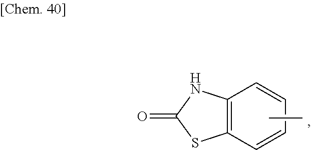

in case that a bond is drawn across a benzene ring, it means that the bond is connected to any substitutable carbon atom which composes the benzene ring. As shown in the formula:

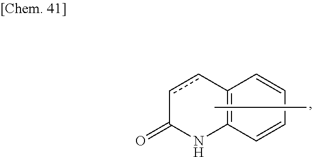

in case that a bond is drawn across both rings of fused ring, it means that the bond is connected to any substitutable carbon or nitrogen atom which composes the both rings.

X is hydrogen atom or fluorine atom, preferably fluorine atom.

R is hydrogen atom or alkyl, preferably hydrogen atom.

R¹ is (1) cyclopropyl which may be optionally substituted with 1 to 3 the same or different halogen atoms, or (2) phenyl which may be optionally substituted with 1 to 3 the same or different halogen atoms; preferably, cyclopropyl, 2-fluorocyclopropyl, or 2,4-difluorophenyl.

R² is hydrogen atom; alkyl which may be optionally substituted with 1 or 2 substituents selected independently from the group consisting of halogen atom and hydroxyl; alkoxy; haloalkoxy; halogen atom; cyano; cyclopropyl; nitro; amino; formyl; alkenyl; or alkynyl, preferably, alkyl, alkoxy, haloalkoxy, chlorine atom, or cyano, more preferably, C₁₋₆ alkyl, C₁₋₆ alkoxy, C₁₋₆ alkoxy substituted with 1 to 3 halogen atoms, chlorine atom, or cyano, even more preferably, methyl, methoxy, or chlorine atom.

The fused heterocyclyl group of formula (A) or (B) includes a fused heterocyclyl group of the formula:

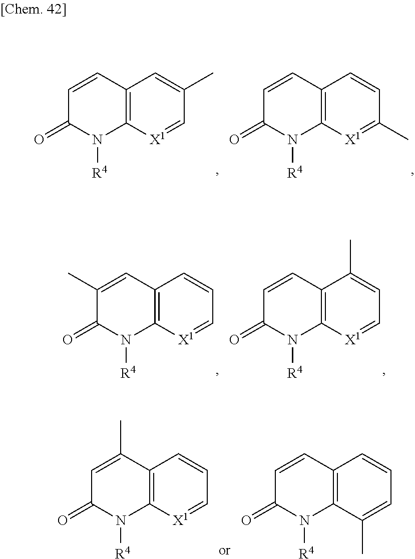

wherein X¹ and R⁴ are as defined above, and said fused heterocyclyl group may be optionally substituted with 1 or 2 substituents selected independently from halogen atom, cyano, nitro, hydroxy, and alkyl.

Preferred examples of the fused heterocyclyl group of formula (A) or (B) include a fused heterocyclyl group of formula:

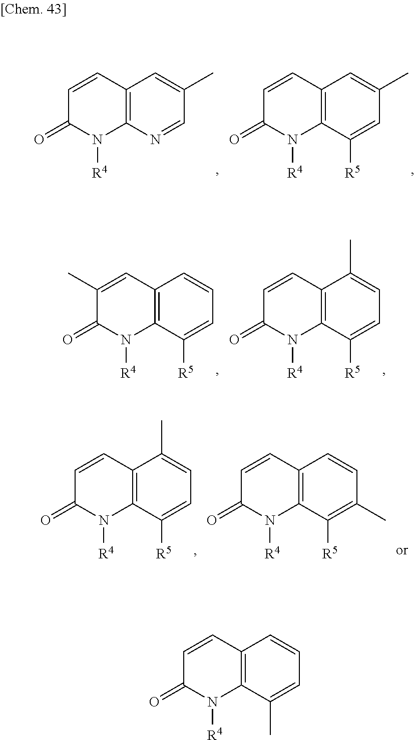

wherein R⁴ and R⁵ are as defined above, and said fused heterocyclyl group may be optionally substituted with 1 or 2 substituents selected independently from halogen atom, cyano, nitro, hydroxy, and alkyl.

Other preferred examples of the fused heterocyclyl group of formula (A) or (B) include a fused heterocyclyl group of formula:

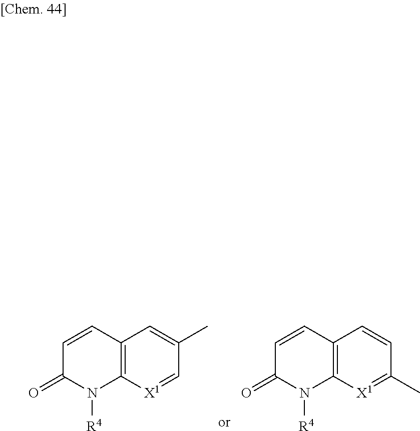

wherein X¹ and R⁴ are as defined above, and said fused heterocyclyl group may be optionally substituted with 1 or 2 substituents selected independently from halogen atom, cyano, nitro, hydroxy, and alkyl.

The group of formula (C) includes a group of the formula:

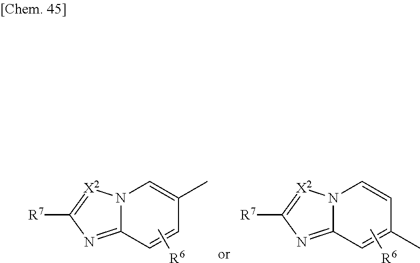

wherein X², R⁶, and R⁷ are as defined above.

Preferred examples of the group of formula (C) include a group of the formula:

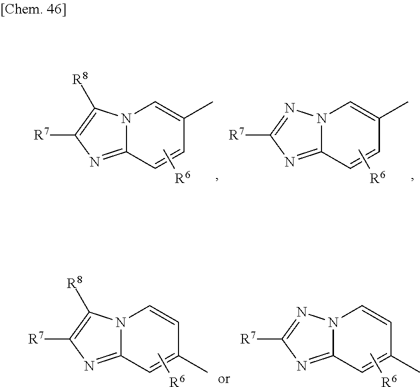

wherein R⁶, R⁷, and R⁸ are as defined above.

In the above formulae, R⁶, R⁷, and R⁸ are independently,

(a) hydrogen atom,

(b) halogen atom,

(c) cyano,

(d) nitro,

(e) amino,

(f) alkyl which may be optionally substituted with 1 to 3 substituents selected independently from the group consisting of halogen atom and amino,

(g) alkenyl,

(h) alkynyl,

(i) aryl,

(j) formyl,

(k) carboxy,

(l) carbamoyl, or

(m) 5- to 10-membered aromatic heterocyclyl group (e.g. pyridyl, triazolyl) which may be optionally substituted with alkyl.

The group of formula (D) or (E) includes a group of the formula:

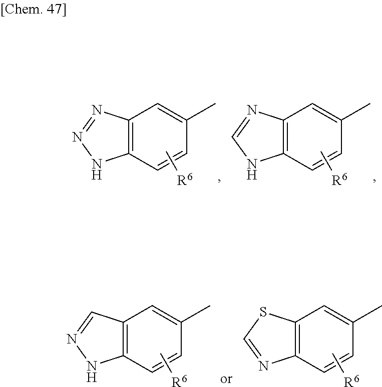

Wherein R⁶ is as defined above. R⁶ is preferably hydrogen atom, halogen atom, nitro, or amino.

Preferably, R³ is 3-pyridyl which may be optionally substituted with 1 or 2 substituents selected independently from the group consisting of the following (a)-(q):

(a) halogen atom,

(b) cyano,

(c) nitro,

(d) hydroxy,

(e) amino,

(f) alkyl which may be optionally substituted with 1 to 3 substituents selected independently from the group consisting of halogen atom, alkylamino, dialkylamino, and hydroxy,

(g) alkenyl,

(h) aryl,

(i) cycloalkyl,

(j) alkoxy,

(k) alkylamino,

(l) dialkylamino,

(m) phenylamino which may be optionally substituted with 1 to 3 the same or different halogen atoms,

(n) cyclic amino group (e.g. 1-piperazinyl, morpholino) which may be optionally substituted with alkoxycarbonyl,

(o) formyl,

(p) carbamoyl, and

(q) 5- to 10-membered aromatic heterocyclyl group (e.g. triazolyl) which may be optionally substituted with alkyl.

More preferably, R³ is a group of the formula:

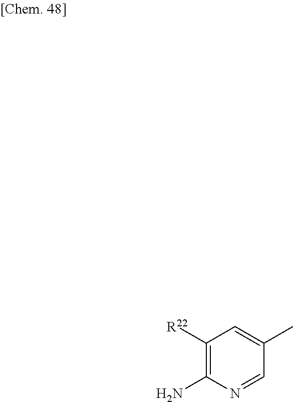

wherein R²² is

(a) halogen atom,

(b) cyano,

(c) nitro,

(d) alkyl which may be optionally substituted with 1 to 3 substituents selected independently from the group consisting of halogen atom, alkylamino, dialkylamino, and hydroxy,

(e) alkenyl,

(f) aryl,

(g) cycloalkyl,

(h) alkoxy,

(i) formyl, or

(j) carbamoyl.

Preferably, R²² is

(a) cyano,

(b) nitro,

(c) aryl,

(d) formyl, or

(e) carbamoyl.

Preferably, R³ is 5-pyrimidinyl substituted with 1 or 2 substituents selected independently from the group consisting of amino, alkylamino, and dialkylamino.

Preferably, R³ is 2-indolyl, 3-indolyl, 5-indolyl, or 6-indolyl, which may be optionally substituted with 1 or 2 substituents selected independently from the group consisting of the following (a)-(j),

(a) halogen atom,

(b) cyano,

(c) nitro,

(d) hydroxy,

(e) alkyl which may be optionally substituted with 1 to 3 substituents selected independently from the group consisting of amino, alkoxycarbonylamino, alkylamino, and dialkylamino,

(f) alkoxy,

(g) formyl,

(h) carboxy, and

(j) amino which may be optionally substituted with 1 or 2 substituents selected independently from the group consisting of the following (i)-(x):

(i) alkoxycarbonyl,

(ii) alkylcarbonyl which may be optionally substituted with a substituent selected from the group consisting of the following (A)-(E):

(A) cycloalkyloxy which may be optionally substituted with 1 to 3 the same or different alkyl,

(B) alkylamino,

(C) dialkylamino,

(D) cyclic amino group (e.g. morpholino, 1-piperazinyl) which may be optionally substituted with alkoxycarbonyl, and

(E) halogen atom,

(iii) phenylcarbonyl which may be optionally substituted with 1 to 3 substituents selected independently from the group consisting of alkyl and alkoxy,

(iv) cycloalkylcarbonyl,

(v) 5- to 10-membered aromatic heterocyclylcarbonyl group (e.g. pyridylcarbonyl) which may be optionally substituted with alkyl optionally-substituted with 1 to 3 the same or different halogen atoms,

(vi) benzylcarbonyl which may be substituted with 1 to 3 substituents selected independently from the group consisting of halogen atom and alkoxy,

(vii) arylsulfonyl which may be optionally substituted with alkoxy,

(viii) cycloalkylalkylsulfonyl (e.g. camphorsulfonyl) which may be optionally substituted with 1 to 3 substituents selected independently from the group consisting of alkyl and oxo,

(ix) 5- to 10-membered aromatic heterocyclylsulfonyl group (e.g. imidazolylsulfonyl) which may be optionally substituted with 1 to 3 the same or different alkyl, and

(x) —C(═N—CN)—SR⁹ wherein R⁹ is alkyl.

More preferably, R³ is 2-indolyl which may be optionally substituted with 1 or 2 substituents selected independently from the group consisting of the following (a)-(j),

(a) halogen atom,

(b) cyano,

(c) nitro,

(d) hydroxy,

(e) alkyl which may be optionally substituted with 1 to 3 substituents selected independently from the group consisting of amino, alkoxycarbonylamino, alkylamino, and dialkylamino,

(f) alkoxy,

(g) formyl,

(h) carboxy, and

(j) amino which may be optionally substituted with 1 or 2 substituents selected independently from the group consisting of the following (i)-(x):

(i) alkoxycarbonyl,

(ii) alkylcarbonyl which may be optionally substituted with a substituent selected from the group consisting of the following (A)-(E):

(A) cycloalkyloxy which may be optionally substituted with 1 to 3 the same or different alkyl,

(B) alkylamino,

(C) dialkylamino,

(D) cyclic amino group (e.g. morpholino, 1-piperazinyl) which may be optionally substituted with alkoxycarbonyl, and

(E) halogen atom,

(iii) phenylcarbonyl which may be optionally substituted with 1 to 3 substituents selected independently from the group consisting of alkyl and alkoxy,

(iv) cycloalkylcarbonyl,

(v) 5- to 10-membered aromatic heterocyclylcarbonyl group (e.g. pyridylcarbonyl) which may be substituted with alkyl optionally-substituted with 1 to 3 the same or different halogen atoms,

(vi) benzylcarbonyl which may be optionally substituted with 1 to 3 substituents selected independently from the group consisting of halogen atoms and alkoxy,

(vii) arylsulfonyl which may be optionally substituted with alkoxy,

(viii) cycloalkylalkylsulfonyl (e.g. camphorsulfonyl) which may be optionally substituted with 1 to 3 substituents selected independently from the group consisting of alkyl and oxo,
(ix) 5- to 10-membered aromatic heterocyclylsulfonyl group (e.g. imidazolylsulfonyl) which may be optionally substituted with 1 to 3 the same or different alkyl, and

(x) —C(═N—CN)—SR⁹ wherein R⁹ is alkyl.

The group of formula (F) or (G) includes a group of the formula:

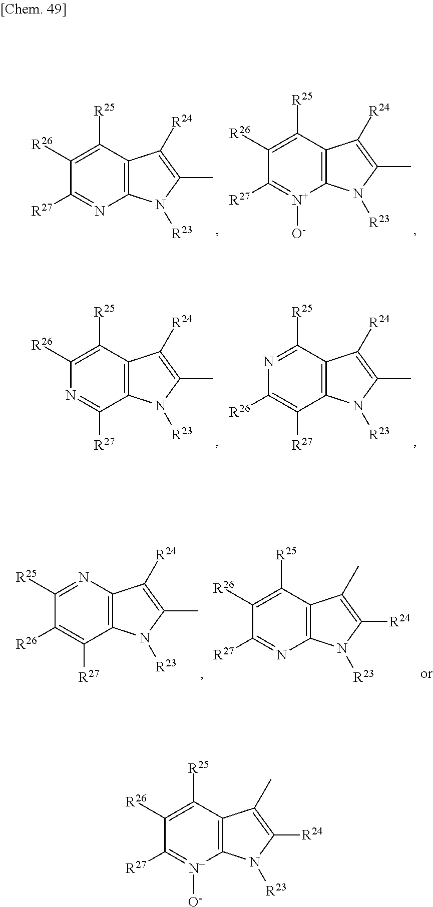

wherein

R²³ is hydrogen atom or alkyl,

R²⁴, R²⁵, R²⁶, and R²⁷ are each independently

(a) hydrogen atom,

(b) cyano, or

(c) nitro.

The group of formula (K) includes a group of the formula:

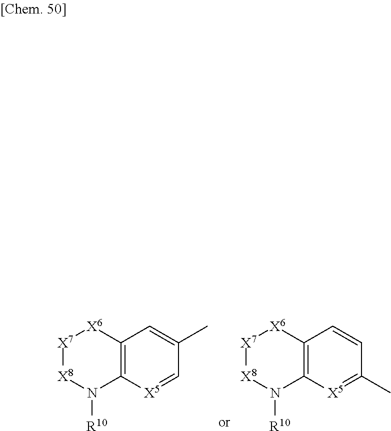

wherein X⁵, X⁶, X⁷, X⁸, and R¹⁰ are as defined above.

The group of formula (K) includes a group of the formula:

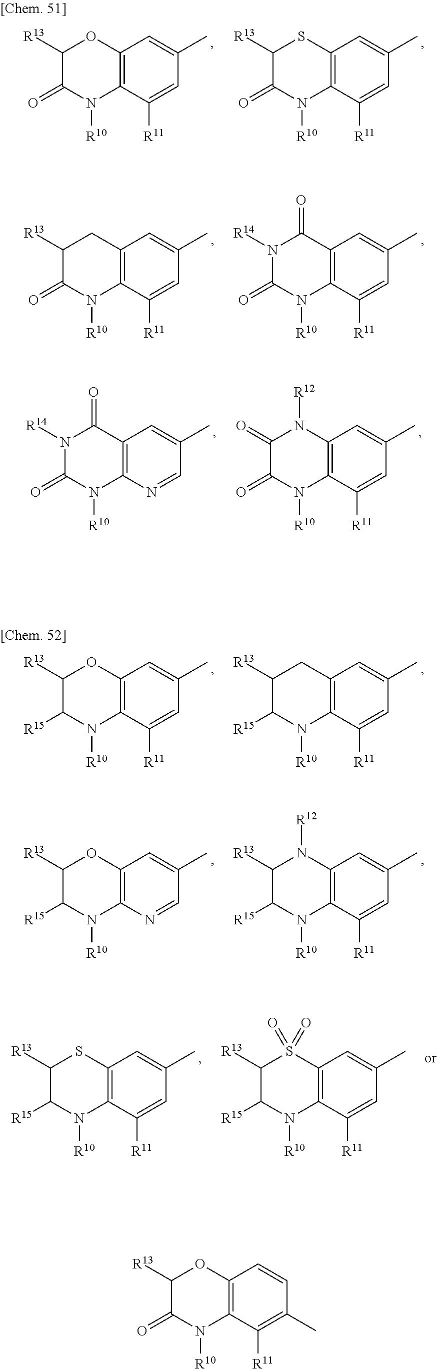

wherein R¹⁰, R¹¹, R¹², R¹³, R¹⁴, and R¹⁵ are as defined above.

When R¹⁰ and R¹¹ are taken together to form 5- or 6-membered ring which may be optionally substituted with alkyl or oxo, preferred examples of the group of formula (K) include a group of the formula:

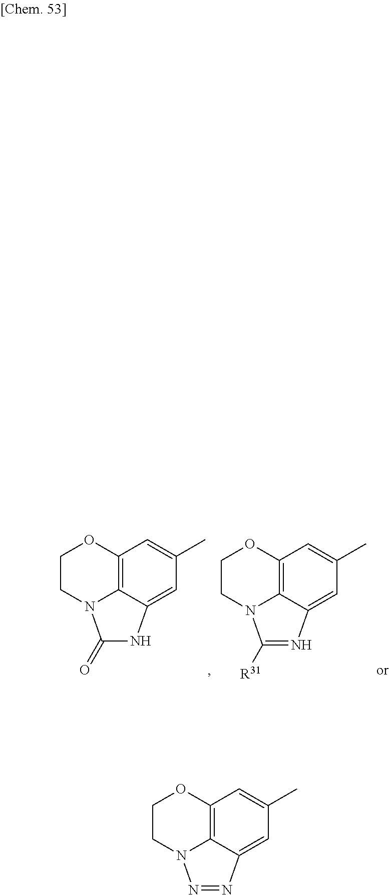

wherein R³¹ is hydrogen atom or alkyl.

When R¹² and R¹³ are taken together to form 5- or 6-membered ring, preferred examples of the group of formula (K) include a group of the formula:

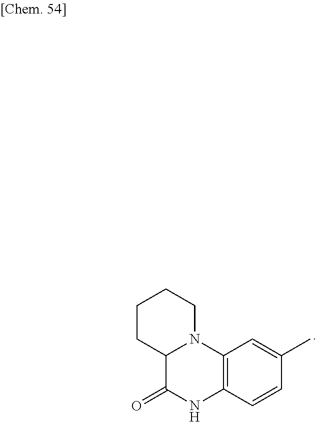 [SMILES: Cc1ccc2c(c1)N1CCCCC1C(=O)N2]

More preferred examples of the group of formula (K) include a group of the formula:

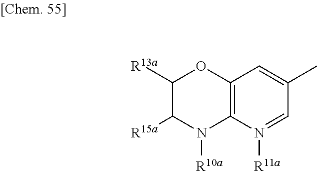

wherein R^{10a} is

(a) hydrogen atom, or

(b) alkyl,

R^{11a}, R^{13a}, and R^{15a} are each independently,

(a) hydrogen atom,

(b) halogen atom,

(c) cyano,

(d) nitro,

(e) amino,

(f) alkylamino,

(g) dialkylamino,

(h) alkyl which may be optionally substituted with hydroxy, or

(i) alkenyl,

R^{10a} and R^{11a} may be taken together to form 5- or 6-membered ring which may be optionally substituted with alkyl or oxo,

provided that not all of R^{10a}, R^{11a}, R^{13a}, and R^{15a} are hydrogen atom.

Preferably, R³ is a group of the formula:

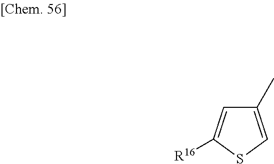

wherein R¹⁶ is

(a) hydrogen atom,

(b) alkyl which may be optionally substituted with 1 to 3 substituents selected from the group consisting of cyano, alkylamino, and dialkylamino,

(c) alkenyl which may be optionally substituted with carboxy,

(d) formyl,

(e) carboxy,

(f) carbamoyl,

(g) —C(R¹⁷)═N—OH wherein R¹⁷ is hydrogen atom, cyano, or hydroxy, or

(h) 5- to 10-membered aromatic heterocyclyl group (e.g. tetrazolyl, pyrrolyl, oxazolyl, benzimidazolyl, triazolyl) which may be optionally substituted with alkyl, alkoxycarbonyl, carboxy, or phenyl.

More preferably, R³ is a group of the formula:

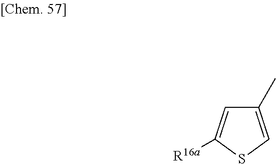

wherein R^{16a} is

(a) alkyl which may be optionally substituted with 1 to 3 substituents selected from the group consisting of cyano, alkylamino, and dialkylamino,

(b) alkenyl which may be optionally substituted with carboxy,

(c) formyl,

(d) carboxy,

(e) carbamoyl,

(f) —C(R¹⁷)═N—OH wherein R¹⁷ is hydrogen atom, cyano, or hydroxy, or

(g) 5- to 10-membered aromatic heterocyclyl group (e.g. tetrazolyl, pyrrolyl, oxazolyl, benzimidazolyl, triazolyl) which may be optionally substituted with alkyl, alkoxycarbonyl, carboxy, or phenyl.

Preferably, R³ is a group of the formula:

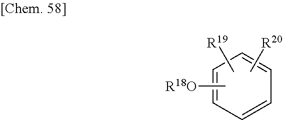

wherein

R¹⁸ is alkyl which may be optionally substituted with 1 to 3 substituents selected independently from the group consisting of halogen atom and phenyl,

R¹⁹ and R²⁰ are each independently,

(a) hydrogen atom,

(b) halogen atom,

(c) cyano,

(d) alkyl which may be optionally substituted with 1 to 3 substituents selected independently from the group consisting of the following (i)-(vii):

(i) halogen atom,

(ii) cyano,

(iii) hydroxy,

(iv) amino,

(v) alkylamino,

(vi) dialkylamino, and

(vii) cyclic amino group (e.g. 1-piperazinyl) which may be optionally substituted with alkyl,

(e) alkoxy,

(f) amino which may be optionally substituted with 1 or 2 substituents selected independently from the following (i)-(iii):

(i) alkylcarbonyl which may be optionally substituted with cyclic amino group (e.g. morpholino),

(ii) alkylsulfonyl, and

(iii) carbamoyl,

(g) carboxy,

(h) alkoxycarbonyl,

(i) carbamoyl which may be optionally substituted with alkyl optionally-substituted with amino, alkylamino, dialkylamino, or alkoxycarbonylamino,

(j) formyl,

(k) 5- to 10-membered aromatic heterocyclyl group (e.g. oxazolyl, benzimidazolyl), or

(l) —CH═N—OR²¹ wherein R²¹ is hydrogen atom, or alkyl which may be optionally substituted with alkylamino or dialkylamino.

More preferably, R³ is a group of the formula:

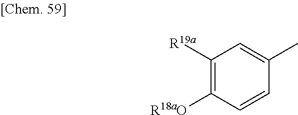

wherein

R^{18a} is alkyl, and

R^{19a} is

(a) halogen atom,

(b) cyano,

(c) alkyl which may be optionally substituted with 1 to 3 substituents selected independently from the group consisting of the following (i)-(vii):

(i) halogen atom,

(ii) cyano,

(iii) hydroxy,

(iv) amino,

(v) alkylamino,

(vi) dialkylamino, and

(vii) cyclic amino group (e.g. 1-piperazinyl) which may be substituted with alkyl,

(d) alkoxy,

(e) amino which may be optionally substituted with 1 or 2 substituents selected independently from the following (i)-(iii):

(i) alkylcarbonyl which may be optionally substituted with cyclic amino group (e.g. morpholino),

(ii) alkylsulfonyl, and

(iii) carbamoyl,

(f) carboxy,

(g) alkoxycarbonyl,

(h) carbamoyl which may be optionally substituted with alkyl optionally-substituted with amino, alkylamino, dialkylamino, or alkoxycarbonylamino,

(i) formyl,

(j) 5- to 10-membered aromatic heterocyclyl group (e.g. oxazolyl, benzimidazolyl), or

(k) —CH═N—OR²¹ wherein R^{2′} is hydrogen atom, or alkyl which may be optionally substituted with alkylamino or dialkylamino.

Preferred examples of Compound (I) are as described below.

(Compound I-1)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom; or

R¹ and R² may be optionally taken together to form —O—CH₂—CH(CH₃)— wherein the oxygen atom is bonded to the phenyl ring in the quinolone ring;

R³ is a fused heterocyclyl group of the formula:

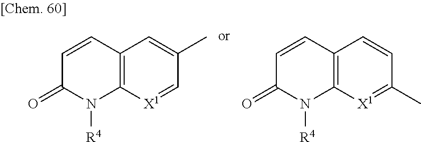

wherein

X¹ is C(R⁵) or N,

R⁴ is hydrogen atom or C₁₋₆ alkyl, and

R⁵ is

(a) hydrogen atom,

(b) halogen atom,

(c) cyano,

(d) nitro,

(e) hydroxy,

(f) C₁₋₆ alkyl which may be optionally substituted with 1 to 3 the same or different halogen atoms,

(g) C₂₋₆ alkynyl,

(h) C₆₋₁₄ aryl, or

(i) C₁₋₆ alkoxy which may be optionally substituted with 1 to 3 the same or different halogen atoms,

when X¹ is C(R⁵), R⁴ and R⁵ may be taken together to form —CH₂—O—(C═O)— wherein the carbonyl is bonded to the phenyl ring in the quinolone ring, said fused heterocyclyl group may be optionally substituted with 1 or 2 substituents selected independently from the group consisting of halogen atom, cyano, nitro, hydroxy, and C₁₋₆ alkyl.

(Compound I-2)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom; or

R¹ and R² may be optionally taken together to form —O—CH₂—CH(CH₃)— wherein the oxygen atom is bonded to the phenyl ring in the quinolone ring; and

R³ is a group of the formula:

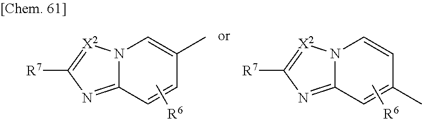

wherein

X² is C(R⁸) or N, and

R⁶, R⁷, and R⁸ are each independently,

(a) hydrogen atom,

(b) halogen atom,

(c) cyano,

(d) nitro,

(e) amino,

(f) C₁₋₆ alkyl which may be optionally substituted with 1 to 3 substituents selected independently from the group consisting of halogen atom and amino,

(g) C₂₋₆ alkenyl,

(h) C₂₋₆ alkynyl,

(i) C₆₋₁₄ aryl,

(j) formyl,

(k) carboxy,

(l) carbamoyl, or

(m) 5- to 10-membered aromatic heterocyclyl group (e.g. pyridyl, triazolyl) which may be optionally substituted with C₁₋₆ alkyl.

(Compound I-3)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

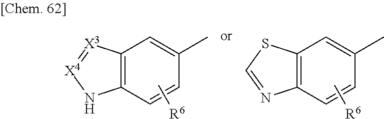

wherein

X³ and X⁴ are N, or

X³ is N, and X⁴ is CH, or

X³ is CH, and X⁴ is N, and

R⁶ is hydrogen atom, halogen atom, nitro, or amino.

(Compound I-4)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom; and

R³ is a group of the formula:

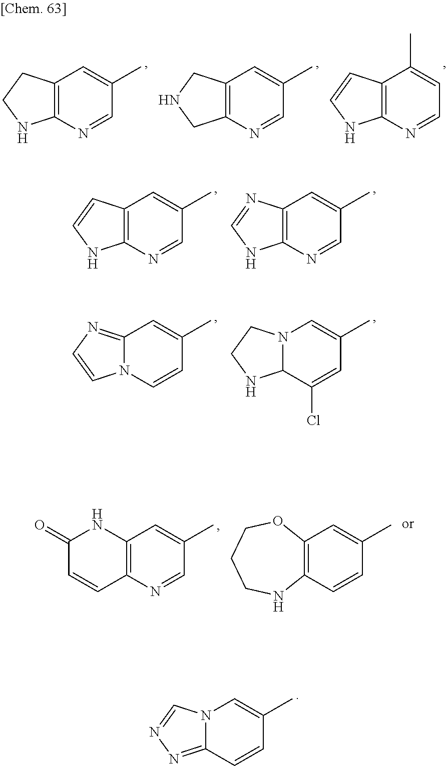

(Compound I-5)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom; or

R¹ and R² may be optionally taken together to form —O—CH₂—CH(CH₃)— wherein the oxygen atom is bonded to the phenyl ring in the quinolone ring; and

R³ is a group of the formula:

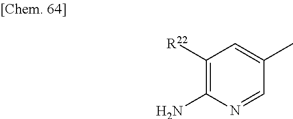

wherein R²² is

(a) halogen atom,

(b) cyano,

(c) nitro,

(d) C₁₋₆ alkyl which may be optionally substituted with 1 to 3 substituents selected independently from the group consisting of halogen atom, C₁₋₆ alkylamino, di(C₁₋₆ alkyl)amino, and hydroxy,

(e) C₂₋₆ alkenyl,

(f) C₆₋₁₄ aryl,

(g) C₃₋₈ cycloalkyl,

(h) C₁₋₆ alkoxy,

(i) formyl, or

(j) carbamoyl.

(Compound I-6)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom; or

R¹ and R² may be optionally taken together to form —O—CH₂—CH(CH₃)— wherein the oxygen atom is bonded to the phenyl ring in the quinolone ring;

R³ is a group of the formula:

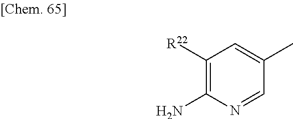

wherein R²² is

(a) cyano,

(b) nitro,

(c) aryl,

(d) formyl, or

(e) carbamoyl.

(Compound I-7)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is 5-pyrimidinyl substituted with 1 or 2 substituents selected independently from the group consisting of amino, C₁₋₆ alkylamino, and di(C₁₋₆ alkyl)amino.

(Compound I-8)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is 2-indolyl which may be optionally substituted with 1 or 2 substituents selected independently from the group consisting of the following (a)-(j):

(a) halogen atom,

(b) cyano,

(c) nitro,

(d) hydroxy,

(e) C₁₋₆ alkyl which may be optionally substituted with 1 to 3 substituents selected independently from the group consisting of amino, C₁₋₆ alkoxy-carbonylamino, C₁₋₆ alkylamino, and di(C₁₋₆ alkyl)amino,

(f) C₁₋₆ alkoxy,

(g) formyl,

(h) carboxy, and

(j) amino which may be optionally substituted with 1 or 2 substituents selected independently from the group consisting of the following (i)-(x):

(i) C₁₋₆ alkoxy-carbonyl,

(ii) C₁₋₆ alkyl-carbonyl which may be optionally substituted with a substituent selected from the group consisting of the following (A)-(E):

(A) C₃₋₈ cycloalkyloxy which may be optionally substituted with 1 to 3 the same or different C₁₋₆ alkyl,

(B) C₁₋₆ alkylamino,

(C) di(C₁₋₆ alkyl)amino,

(D) cyclic amino group (e.g. morpholino, 1-piperazinyl) which may be optionally substituted with C₁₋₆ alkoxy-carbonyl, and

(E) halogen atom,

(iii) phenylcarbonyl which may be optionally substituted with 1 to 3 substituents selected independently from the group consisting of C₁₋₆ alkyl and C₁₋₆ alkoxy,

(iv) C₃ cycloalkyl-carbonyl,

(v) 5- to 10-membered aromatic heterocyclylcarbonyl group (e.g. pyridylcarbonyl) which may be optionally substituted with C₁₋₆ alkyl optionally-substituted with 1 to 3 the same or different halogen atoms,

(vi) benzylcarbonyl which may be optionally substituted with 1 to 3 substituents selected independently from the group consisting of halogen atom and C₁₋₆ alkoxy,

(vii) C₆₋₁₄ arylsulfonyl which may be substituted with C₁₋₆ alkoxy,

(viii) C₃₋₈ cycloalkyl-C₁₋₆ alkylsulfonyl (e.g. camphorsulfonyl) which may be optionally substituted with 1 to 3 substituents selected independently from the group consisting of C₁₋₆ alkyl and oxo,

(ix) 5- to 10-membered aromatic heterocyclylsulfonyl group (e.g. imidazolylsulfonyl) which may be optionally substituted with 1 to 3 the same or different C₁₋₆ alkyl, and

(x) —C(═N—CN)—SR⁹ wherein R⁹ is alkyl.

(Compound I-9)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

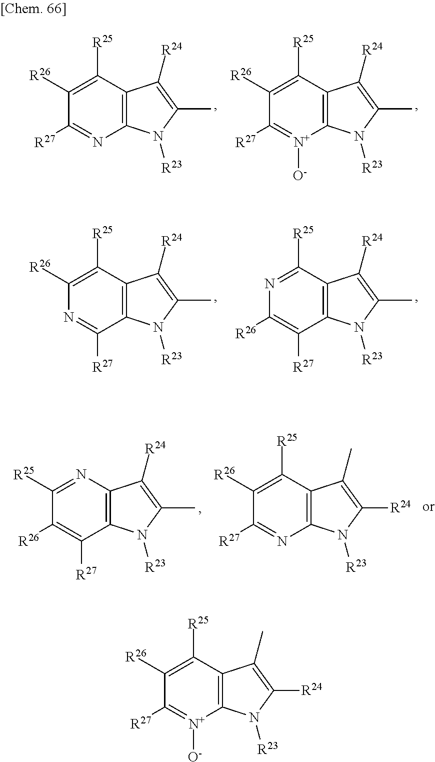

wherein

R²³ is hydrogen atom or C₁₋₆ alkyl,

R²⁴, R²⁵, R²⁶, and R²⁷ are each independently,

(a) hydrogen atom,

(b) cyano, or

(c) nitro.

(Compound I-10)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom; and

R³ is a group of the formula:

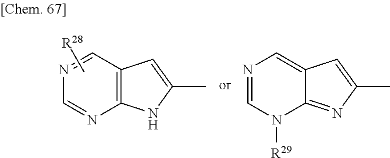

wherein

R²⁸ is hydrogen atom or hydroxyl, and

R²⁹ is hydrogen atom or C₁₋₆ alkyl.

(Compound I-11)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

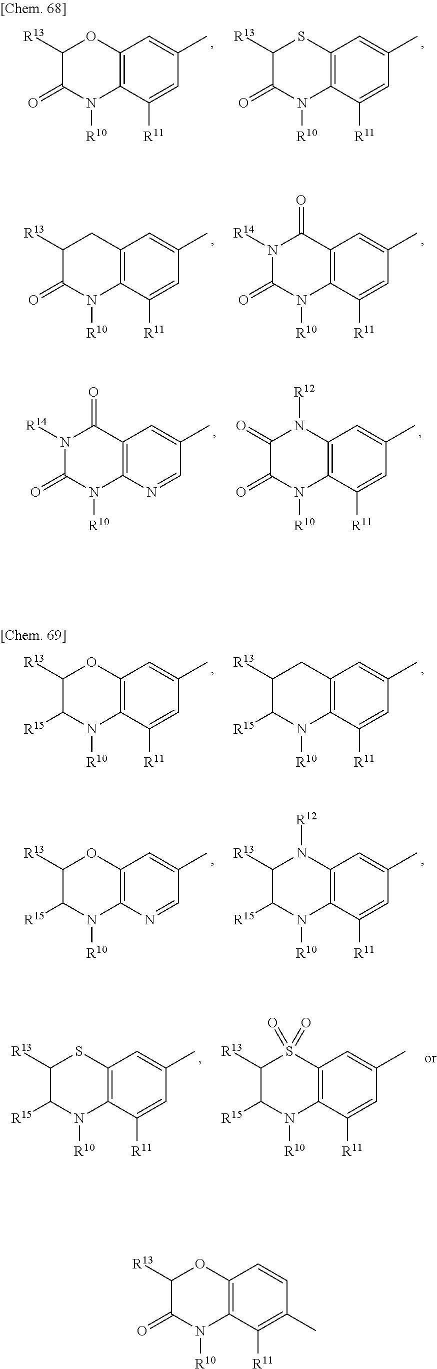

wherein

R¹⁰, R¹², and R¹⁴ are each independently,

(a) hydrogen atom, or

(b) C₁₋₆ alkyl,

R¹¹, R¹³, and R¹⁵ are each independently,

(a) hydrogen atom,

(b) halogen atom,

(c) cyano,

(d) nitro,

(e) amino,

(f) C₁₋₆ alkylamino,

(g) di(C₁₋₆ alkyl)amino,

(h) C₁₋₆ alkyl which may be optionally substituted with hydroxy, or

(i) C₂₋₆ alkenyl, or

R¹⁰ and R¹¹ may be optionally taken together to form —(C═O)—NH—, —C(R³¹)═N—, or —N═N— wherein R³¹ is hydrogen atom or C₁₋₆ alkyl, and the nitrogen atom is bonded to the phenyl ring in the fused ring, or

R¹² and R¹³ may be optionally taken together to form —(CH₂)₄—.

(Compound I-12)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

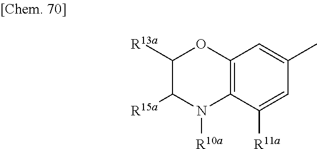

wherein R^{10a} is

(a) hydrogen atom, or

(b) C₁₋₆ alkyl, and

R^{11a}, R^{13a}, and R^{15a} are each independently,

(a) hydrogen atom,

(b) halogen atom,

(c) cyano,

(d) nitro,

(e) amino,

(f) C₁₋₆ alkylamino,

(g) di(C₁₋₆ alkyl)amino,

(h) C₁₋₆ alkyl which may be optionally substituted with hydroxy, or

(i) C₂₋₆ alkenyl,

provided that not all of R^{10a}, R^{11a}, R^{13a}, and R^{15a} are hydrogen atom.

(Compound I-13)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

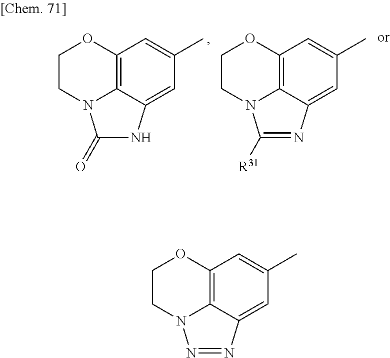

wherein R³¹ is hydrogen atom or C₁₋₆ alkyl.

(Compound I-14)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

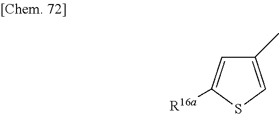

wherein R^{16a} is

(a) C₁₋₆ alkyl which may be optionally substituted with 1 to 3 substituents selected from the group consisting of cyano, C₁₋₆ alkylamino, and di(C₁₋₆ alkyl)amino,

(b) C₂₋₆ alkenyl which may be optionally substituted with carboxy,

(c) formyl,

(d) carboxy,

(e) carbamoyl,

(f) —C(R¹⁷)═N—OH wherein R¹⁷ is hydrogen atom, cyano, or hydroxy, or

(g) 5- to 10-membered aromatic heterocyclyl group (e.g. tetrazolyl, pyrrolyl, oxazolyl, benzimidazolyl, triazolyl) which may be optionally substituted with C₁₋₆ alkyl, C₁₋₆ alkoxy-carbonyl, carboxy, or phenyl.

(Compound I-15)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

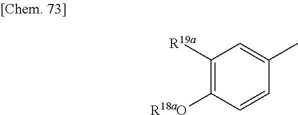

wherein

R^{18a} is C₁₋₆ alkyl, and

R^{19a} is

(a) halogen atom,

(b) cyano,

(c) C₁₋₆ alkyl which may be optionally substituted with 1 to 3 substituents selected independently from the group consisting of the following (i)-(vii):

(i) halogen atom,

(ii) cyano,

(iii) hydroxy,

(iv) amino,

(v) C₁₋₆ alkylamino,

(vi) di(C₁₋₆ alkyl)amino, and

(vii) C₁₋₆ cyclic amino group (e.g. 1-piperazinyl) which may be optionally substituted with alkyl,

(d) C₁₋₆ alkoxy,

(e) amino which may be optionally substituted with 1 or 2 substituents selected independently from the group consisting of the following (i)-(iii):

(i) C₁₋₆ alkyl-carbonyl which may be optionally substituted with cyclic amino group (e.g. morpholino),

(ii) C₁₋₆ alkylsulfonyl, and

(iii) carbamoyl,

(f) carboxy,

(g) C₁₋₆ alkoxy-carbonyl,

(h) carbamoyl which may be optionally substituted with C₁₋₆ alkyl optionally-substituted with amino, C₁₋₆ alkylamino, di(C₁₋₆ alkyl)amino, or C₁₋₆ alkoxy-carbonylamino,

(i) formyl,

(j) 5- to 10-membered aromatic heterocyclyl group (e.g. oxazolyl, benzimidazolyl), or

(k) —CH═N—OR²¹ wherein R²¹ is hydrogen atom, or C₁₋₆ alkyl which may be optionally substituted with C₁₋₆ alkylamino or di(C₁₋₆ alkyl)amino.

(Compound I-16)

A compound of formula (I) or a salt thereof, wherein

R is hydrogen atom;

R¹ is cyclopropyl, 2-fluorocyclopropyl, or 2,4-difluorophenyl;

R² is C₁₋₆ alkyl (e.g. methyl), C₁₋₆ alkoxy (e.g. methoxy), or chlorine atom;

R³ is a group of the formula:

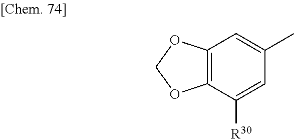

wherein R³⁰ is

(a) hydrogen atom,

(b) halogen atom,

(c) cyano,

(d) C₁₋₆ alkyl which may be optionally substituted with 1 to 3 substituents selected from the group consisting of halogen atoms and hydroxy,

(e) C₂₋₆ alkenyl,

(f) C₂₋₆ alkynyl,

(g) C₁₋₆ alkoxy,

(h) formyl, or

(i) —CH═N—OH.

A compound selected from the group consisting of formula:

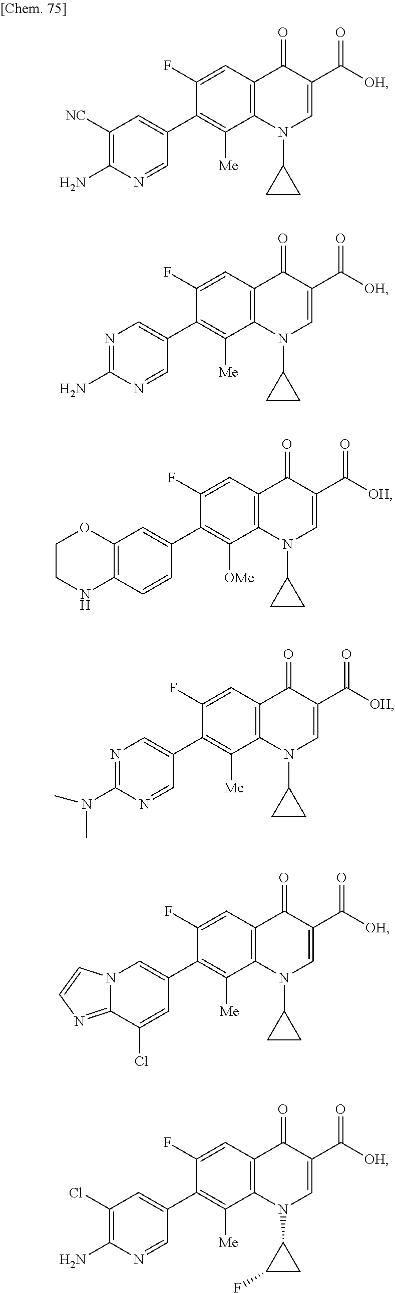

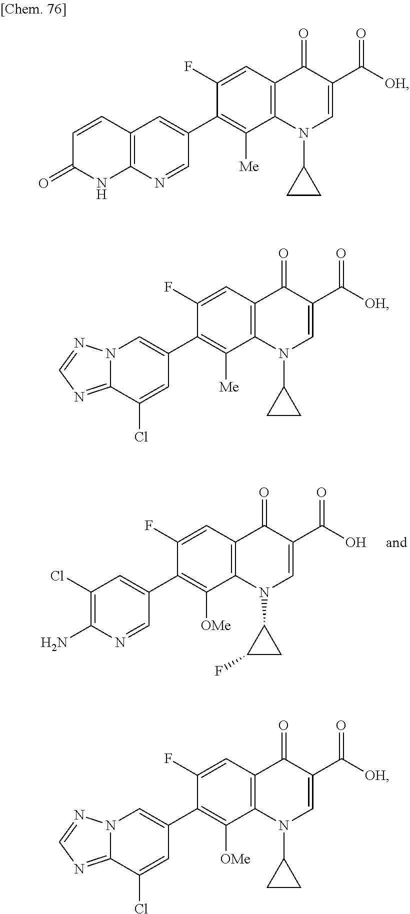 [SMILES: Cc1c(-c2cnc3[nH]c(=O)ccc3c2)c(F)cc2c(=O)c(C(=O)O)cn(C3CC3)c12 ; Cc1c(-c2cc(Cl)c3ncnn3c2)c(F)cc2c(=O)c(C(=O)O)cn(C3CC3)c12 ; COc1c(-c2cnc(N)c(Cl)c2)c(F)cc2c(=O)c(C(=O)O)cn([C@@H]3C[C@@H]3F)c12 ; "and" ; COc1c(-c2cc(Cl)c3ncnn3c2)c(F)cc2c(=O)c(C(=O)O)cn(C3CC3)c12]

or a salt thereof.

The quinolone compounds of the present invention are disclosed in Patent Literature 1 about their preparation processes and the antibacterial activity against Clostridium difficile.

The compound of the present invention may be in a form of hydrate and/or solvate, thus the compound of the present invention also encompasses such hydrate thereof and solvate thereof.

In addition, the compound of the present invention in which any one or more ¹H atoms are replaced by ²H(D) atoms is within the scope of the present invention.

When the compound of the present invention or a pharmaceutically acceptable salt thereof is obtained as a crystal, the crystal may include crystalline polymorph. Thus, the present invention also encompasses such crystalline polymorph.

The “pharmaceutically acceptable salt” includes, as an acid addition salt, a salt with inorganic acid such as hydrochloride, hydrobromide, hydroiodide, sulfate, perchlorate, and phosphate; a salt with organic acid such as oxalate, malonate, maleate, fumarate, lactate, malate, citrate, tartrate, benzoate, trifluoroacetate, acetate, methanesulfonate, p-toluenesulfonate, and trifluoromethanesulfonate; and a salt with amino acid such as glutamate and aspartate; and as a salt with a base, an alkali metal salt such as sodium salt and potassium salt; alkaline-earth metal salt such as calcium salt; and an ammonium salt.

The “inflammatory bowel disease” used herein is a collective term of chronic diseases causing inflammation mainly in gastrointestinal tract, including Crohn's disease, ulcerative colitis, ileositis, diverticulitis, irritable bowel syndrome and traveler's diarrhea; especially Crohn's disease and ulcerative colitis.

The “Crohn's disease” is a disease of unknown cause, that can occur in any part of the digestive tract from oral cavity to anus. It causes the formulation of discontinuous edema or ulcer mainly in small intestine/large intestine, and then causes a distinctive pathology such as intestinal stenosis and fistula. The specific symptom includes various symptoms such as abdominal pain, diarrhea, hematochezia, fever, and weight loss, as well as various complications such as pain and puffiness around the anus. The “ulcerative colitis” is a chronic inflammatory disease of unknown cause that occurs in large intestine, which causes ulcer and erosion in mucosa of large intestine. The lesion area starts mainly at rectum, and sometimes spreads to the whole of large intestine. The symptom includes hematochezia, mucous stool, diarrhea, and abdominal pain. It is a chronic disease repeating remission and relapse.

The present compound may be administered via any route selected from oral administration, parenteral administration and rectal administration. Oral administration and rectal administration are preferable. The daily dose depends on the compound, administration route, condition of patient, age of patient, etc. In case of oral administration, for example, it may be generally administered in a dose of about 0.01 mg-about 100 mg, preferably about 0.1 mg-about 50 mg, more preferably about 2.5 mg-about 20 mg, even more preferably about 5 mg-about 10 mg, per kg of human or mammal's body weight, in one to several portions. For example, the daily dose of human includes about 0.5 mg-about 6000 mg, preferably about 30 mg-about 3000 mg, more preferably about 150 mg-about 1200 mg, even more preferably about 300 mg-about 600 mg.

The dosage form includes tablet, capsule, granule, powder, syrup, suspension, injection, suppository, eye drop, ointment, endermic liniment, patch, inhalant, and enema. These dosage forms can be prepared in conventional means. As for liquid formulation, it may be dissolved or suspended with water, a suitable aqueous solution or a suitable solvent, when used. As for tablet and granule, they may be coated in well-known manner. The dosage form may be prepared in known manner with pharmaceutically acceptable additives.

The additives used herein include, according to the intended use, excipients, disintegrating agents, binders, fluidizer, lubricants, coating agents, colorants, solubilizers, solubilizing agents, thickeners, dispersants, stabilizing agents, sweeteners, and flavors. For example, they include lactose, mannitol, calcium hydrogen phosphate, microcrystalline cellulose, low substituted hydroxypropylcellulose, cornstarch, partly pregelatinized starch, carmellose calcium, croscarmellose sodium, crospovidone, sodium starch glycolate, hydroxypropylcellulose, hydroxypropyl methylcellulose, polyvinyl alcohol, light anhydrous silicic acid, magnesium stearate, calcium stearate, sodium stearyl fumarate, polyethylene glycol, propylene glycol, titanium oxide, talc, iron sesquioxide, and yellow ferric oxide.

When the present compound is formulated into a single dosage form, the present compound may be contained, for example, in 0.1-85% by weight per the whole composition of the dosage from. Preferably, the present compound is contained in 10-70% by weight per the whole composition of the dosage from.

In addition, the present compound may be used in combination with another drug or as a combination with another drug in order to enhance the effect and/or relieving side effects. The other drug which can be used in combination includes, for example, 5-aminosalicylic acid (5-ASA) and budesonide.

### EXAMPLES

The present invention is explained in more detail in the following by referring to Examples, however, the scope of the present invention is not limited thereto.

In Examples 1 to 8 below, 7-(6-amino-5-cyanopyridin-3-yl)-1-cyclopropyl-6-fluoro-1,4-dihydro-8-methyl-4-oxo-3-quinoline-carboxylic acid was used as the present compound.

### Example 1. Effect to Crohn's Disease Mouse Model

Using a naive T cell transfer model of colitis which has been reported as Crohn's disease model, the preventive effect (Test 1) or therapeutic effect (Test 2) of the present compound was studied, and also the comparative study with antibacterial agents (ciprofloxacin (CPFX), rifaximin (RFX)) and an anti-TNF-α antibody was carried out (Test 3).

(Method)

From spleen of Balb/c mouse, naive T cell (CD4+CD62L+CD44−) was isolated with antibody-coated magnetic beads. The isolated naive T cell (5×10⁵ cells/mouse) was transplanted in abdominal cavity of SCID mouse which is a severe combined immunodeficiency animal.

The present compound was orally administered to the mouse once a day at each dose defined in Table 1 below, from the first day of the transplantation for Test 1, or from the 2nd week after the transplantation for Test 2, until 5 weeks after the transplantation.

TABLE 1

| | Test Group | Dose | N |
|---|---|---|---|
| 1 | Normal (non-transplantation group) | — | 10 |
| 2 | Vehicle (solvent control group) | 0 mg/kg | 10 |
| 3 | The present compound (2.5) | 2.5 mg/kg | 10 |
| 4 | The present compound (5) | 5 mg/kg | 10 |
| 5 | The present compound (10) | 10 mg/kg | 10 |
| 6 | The present compound (20) | 20 mg/kg | 10 |

As for Test 3, the present compound, CPFX, and RFX were orally administered to the mouse once a day at each dose defined in Table 2 below, from the 2nd week after the transplantation, for 2 weeks. The anti-TNF-α antibody was intraperitoneally administered to the mouse twice a week at each dose defined in Table 2 below, from the 2nd week after the transplantation, for 2 weeks.

TABLE 2

| | Test Group | Dose | N |
|---|---|---|---|
| 1 | Normal | — | 10 |
| 2 | Vehicle | 0 mg/kg | 10 |
| 3 | anti-TNF-α antibody (25) | 25 mg/kg | 10 |
| 4 | The present compound (10) | 10 mg/kg | 10 |
| 5 | CPFX (10) | 10 mg/kg | 10 |
| 6 | RFX (20) | 10 mg/kg | 10 |

At 5th week for Tests 1 and 2, and at 4th week for Test 3, the large intestine was removed, and the length and weight of the large intestine were measured. In addition, the large intestine was fixed with formalin to carry out pathological analysis.

(Histological Scoring)

From the removed large intestine, the anus part, the center part, and the cecal part were cut out each in about 0.5 cm, and each part was fixed with formalin. The paraffin block thereof was prepared, sliced, and subjected to H&E staining. The H&E stained slice was examined with a microscope to score it histologically. The histological scoring was carried out by scoring the inflammatory cell infiltrate, the Goblet cell loss, and the hyperplasia of mucosal epithelium, based on the standard (0-4) defined in Table 3 below.

TABLE 3

| | Score | Description of Lesions |
|---|---|---|
| Inflammatory | 0 | None |
| cell | 1 | Minimal: Focal Infiltrate in the |
| infiltrate | | Mucosa |
| | 2 | Mild: Diffuse and Extensive |
| | | Infiltrate in the Mucosa |
| | 3 | Moderate: Infiltrate in the |
| | | Mucosa and Submucosa |
| . | 4 | Severe: Transmural Infiltrate |
| Hyperplasia | 0 | None: 0 < ratio < 1 |
| of mucosal | 1 | Minimal: 1 < ratio ≤ 1.5-fold |
| epithelium | 2 | Mild: 1.5 < ratio ≤ 2-fold |
| | 3 | Moderate: 2 < ratio ≤ 3-fold |
| | 4 | Severe: ratio > 3-fold |
| Goblet cell | 0 | None: almost the same as Normal |
| loss | 1 | Minimal: about less than 25% of |
| | | Normal |
| | 2 | Mild: about less than 50% of |
| | | Normal |
| | 3 | Moderate: about less than 75% of |
| | | Normal |
| | 4 | Severe: about more than 75% of |
| | | Normal |

(Result of Test 1)

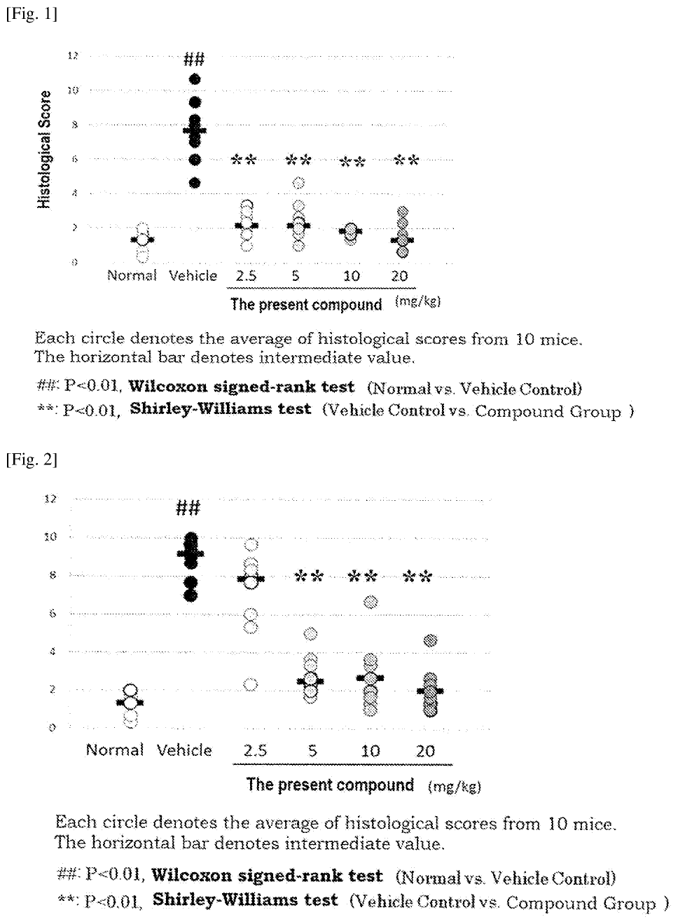

The result is shown in FIG. 1. The histological score of the solvent control group (Vehicle) which was examined 5 weeks after the transplantation of naive T cell was significantly increased, compared with the score of the non-transplantation group (Normal). All of the present compound groups (2.5 mg/kg or more) inhibited the increase of the histological score, compared with the solvent control group. Thus, it has been found that the prevention treatment of the present compound (2.5 mg/kg or more) can strongly inhibit the gastrointestinal tract inflammation in a mouse model suffering from Crohn's disease, which was the same level as Normal group.

(Result of Test 2)

The result is shown in FIG. 2. The histological score of the solvent control group (Vehicle) which was examined 5 weeks after the transplantation of naive T cell was significantly increased, compared with the score of the non-transplantation group (Normal). It has been found that the present compound groups (5 mg/kg or more) significantly inhibited the increase of the histological score, compared with the solvent control group. Thus, it has been found that the intervention treatment of the present compound (5 mg/kg or more) can strongly inhibit the gastrointestinal tract inflammation in a mouse model suffering from Crohn's disease, which was the same level as Normal group.

(Result of Test 3)

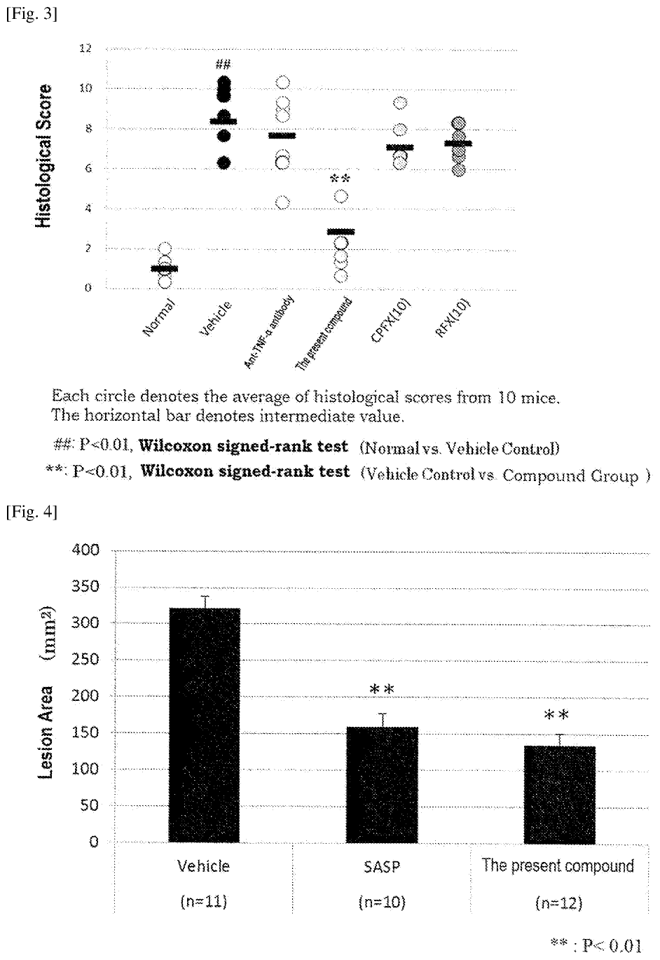

The result is shown in FIG. 3. The large intestine to which naive T cell was transplanted 4 weeks ago presented with obvious inflammatory symptom such as invasion of inflammatory cell and hyperplasia of mucosal epithelium, and showed a significant increase of the histological score. For this colitis, only the present compound showed a significant effect to inhibit the increase of the histological score, and the other drugs did not show such inhibitory effect. Thus, for the gastrointestinal tract inflammation in a mouse model suffering from Crohn's disease, the therapeutic administration of antibacterial agents (CPFX, RFX), and anti-TNF-α antibody showed little or no inhibitory effect, while the therapeutic administration of the present compound (10 mg/kg) showed potent inhibitory effect at the same level as Normal group.

### Example 2. Effect for TNBS-Induced Enteritis Model

An enteritis animal model induced with 2,4,6-trinitrobenzenesulfonic acid (TNBS) is widely used as Crohn's disease model since the inflammatory finding is pathologically similar to that of human Crohn's disease. In addition, the enteritis model was used in the non-clinical study of mesalazine that is a medicament for treating Crohn's disease. Thus, this enteritis model was used as Crohn's disease model for evaluating the present compound.

(Method)

Rats were made to be fasted for 24 or 48 hours, and the body weight were measured. The rats were laparotomized under isoflurane anesthesia, and 50% ethanol (0.25 mL) containing 60 mg/mL 2,4,6-trinitrobenzenesulfonic acid (TNBS) was injected into the large intestine from the cecum side toward the anal side to induce the injury. Each group composed of 12 rats was set, and vehicle, the present compound, or salazosulfapyridine (SASP) which is an already-existing drug for treating inflammatory bowel disease were administered for 7 days from the first day after the injection of TNBS. On the 8th day, each rat was laparotomized under anesthesia and killed by exsanguination. The large intestine was removed from the anus to the cecum, the large intestine was longitudinally incised, and the incised large intestine was washed with a cold saline. The lesion area of the large intestine was photographed with a digital camera. The area of the lesion area in the photograph of the large intestine was measured using image analyzing program.

(Result)

Each compound was orally administered at a dose of 100 mg/kg twice a day from one day after inducing enteritis with TNBS. Each area of the lesion area on the 8th day was compared. The results are shown in FIG. 4. Significant difference were observed between the vehicle control group and SASP group or the present compound group (P<0.01, t-test). In the control group or SASP group, one or two rats were died during the treatment, respectively. The present compound decreased the number of death, compared with SASP, and significantly inhibited the area of lesion, compared with the vehicle control.

### Example 3. Action to Inhibit TNF-α Production

Using the present compound, ciprofloxacin (CPFX), rifaximin (RFX), and 5-aminosalicylic acid (5-ASA), the inhibitory action for the TNF-α production was studied with human peripheral blood cells.

(Test Method)

Each test sample and control sample were prepared as shown in Table 4 below.

TABLE 4

| | Test compound | Concentration | N |
|---|---|---|---|
| 1 | the present compound | 1-100 μmol/L | 3 |
| 2 | CPFX | 3-fold serial | |
| 3 | RFX | dilution | |
| 4 | 5-ASA | | |
| 5 | prednisolone | 0.3 μmol/L | |

The LPS solution which was adjusted to 2 μg/mL with RPMI 1640 medium was added to 48-well plate in the amount of 250 μL/well. The each diluted solution of each test compound and the diluted solution of the solvent control were added to 3 wells for each concentration in the amount of 50 μL/well, 4-fold diluted solution of human peripheral blood was added to each well (200 μL/well), and the wells were incubated at 37° C. in 5% CO₂ for 24 hours. As LPS (−) control, RPMI 1640 medium without added LPS was prepared in the same manner. The final concentrations of each test sample were adjusted to 0, 1, 3, 10, 30, and 100 μM, and the final concentration of prednisolone was adjusted to 0.3 μM. 24 hours later from the incubation, each supernatant was collected and stored at −80° C. until the measurement of the TNF-α concentration.

(Measurement of TNF-α Concentration)

The TNF-α concentration was measured by ELISA with Human TNF-alpha Quantikine ELISA Kit (R & D Systems). Each sample was unfrozen at room temperature, and diluted with Calibrator Diluent RD6-35 (1×) of the kit by 10-fold. The 10-fold diluted supernatant and the TNF-α standard solution were added on TNF-α microplate, and reacted according to the operating manual of the kit. After the coloring, the absorbance at wave length of 450 nm was measured with a microplate reader (Multiskan FC; Thermo Fisher SCIENTIFIC), and the calibration curve was prepared with an analysis software (Skanit Software 3.1.0.4 RE for Multiskan FC (ja)) to read the TNF-α concentration of each sample. Calibration curve used a 4-parameter logistic curve.

(Statistical Analysis Method)

The inhibition rates were presented as the mean±standard deviation using Microsoft Excel 2010. For each blood donor, the TNF-α production rate under each compound concentration was calculated with the TNF-α concentration in the supernatant of the solvent group as 100%.

(Result)

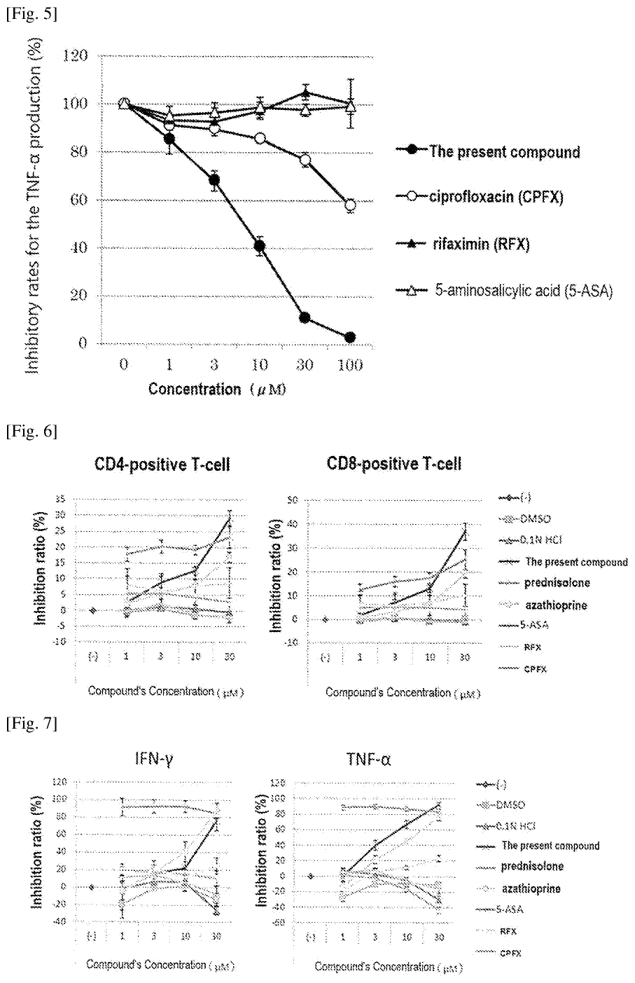

The inhibitory rates for the TNF-α production of the present compound and the comparative compounds are shown in FIG. 5. The results are averages of the measurements obtained from the human peripheral blood of three volunteers.

The present compound inhibited TNF-alpha production in a dose-dependent manner, and 100 μM of the present compound completely inhibited the production. On the other hand, RFX and 5-ASA had no inhibitory activity in all concentrations. CPFX showed a dose-dependent inhibitory effect at a concentration of 30 μM or more, but even at 100 μM, the inhibitory effect was less than 50%. According to the obtained results, IC₅₀ of the present compound was calculated to be 6.25 μM. Each IC₅₀ of the comparative compounds was not able to be calculated because the inhibitory effect was too weak. Thus, among the tested compounds, only the present compound showed a potent inhibitory activity for the TNF-α production.

### Example 4. Action to Inhibit the Activation of T Cell

The action on T-cell response which is thought to be deeply involved in inflammatory bowel disease was studied with human peripheral blood mononuclear cells.

(Preparing PBMC)

Peripheral blood (30 mL) derived from a healthy adult was layered on a lymphocyte tube Leucosep™ containing Lymphoprep (15 mL), and the tube was centrifuged at room temperature at 2000 rpm for 20 minutes. After the centrifugation, the upper 5 mL of the plasma was removed from the peripheral blood mononuclear cell (PBMC) layer with an aspirator, and the left solution containing the PBMC layer was collected into a new 50 mL tube. To the collected solution was added an equal amount of Hanks' balanced salt solution (HBSS) to dilute the cell suspension. The diluted solution was centrifuged at 4° C. at 1800 rpm for 5 minutes. After the centrifugation, the supernatant was discarded, the rest was suspended in 10 mL of AIM-V. The suspension was centrifuged at 4° C. at 1500 rpm for 5 minutes, and the supernatant was discarded. The same washing process was repeated one more time. After the centrifugation, the supernatant was discarded, and the obtained cell was suspended in 5% FBS-containing AIM-V to be used in the following tests.

(Evaluation of T-Cell Activation and Evaluation of Cytokine Production)

To 96-well round-bottom plate were added 100 μL of medium containing the compound, 100 μL (0.5×10⁵ beads) of anti-CD3/CD28/CD2-loaded bead suspension (Miltenyi), and 100 μL (1×10⁵ cells) of PBMC suspension. The mixture was incubated at 37° C. in 5% CO₂ for 3 days. After the centrifugation, the culture supernatant was collected, and each concentration of cytokines (IFN-γ, TNF-α) in the culture supernatant was measured by ELISA. And, the plate which was left after the collection of the culture supernatant was centrifuged to remove the supernatant, and the obtained cells were stained with PE labeled anti-human CD25 antibody, BV421 labeled antihuman CD8a antibody, BV510 labeled anti-human CD4 antibody. The antibody-stained cells were analyzed with a flow cytometer (BD FACSVerse) to evaluate the rate of the activated T cell (CD25+cell). The number of wells to be evaluated was 3 wells per PBMC for each condition.

(Evaluation of T-Cell Proliferation)

To 24-well plate were added 1 mL of AIM-V containing 5% FBS, 0.5 mL (1×10⁶ beads) of anti-CD3/CD28/CD2-loaded bead suspension (Miltenyi), and 0.5 mL (2×10⁶ cells) of PBMC suspension. The mixture was incubated at 37° C. in 5% CO₂ for 3 days. After 3 days, the cells were collected, washed with 10 mL of PBS(−) twice, and stained with CFSE. To 96-well round-bottom plate were added 100 μL of medium containing the compound, 100 μL (0.25×10⁵ beads) of anti-CD3/CD28/CD2-loaded bead suspension (Miltenyi), and 100 μL (0.5×10⁵ cells) of CFSE-stained PBMC suspension. The mixture was incubated at 37° C. in 5% CO₂ for 2 days. After 2 days, the plate incubating the cells was centrifuged to remove the supernatant, and the obtained cells were stained with BV421 labeled anti-human CD8a antibody and BV510 labeled antihuman CD4 antibody. The antibody-stained cells were analyzed with a flow cytometer (BD FACSVerse) to evaluate the fluorescence intensity of CFSE (MFI (CFSE)). The number of wells to be evaluated was 3 wells per PBMC for each condition.

(Result)

Rate of Activated T Cell

The effect of the present compound for activating T cell was studied with 6 people's human PBMC. The rates of the activated T cell in CD4-positive T-cell and CD8-positive T-cell were calculated according to the following formula to obtain their inhibition ratios, and the results were shown in FIG. 6. The assay was carried out by two-way Dunnett, based on DMSO group as a control.

Inhibition ratio (%)=100−((“Rate of each activated T cell”−“Average of rate of activated T cell without stimulus/compound”)/(“Average of rate of activated T cell without compound”−“Average of rate of activated T cell without stimulus/compound”))×100

As for both of CD4-positive T-cell and CD8-positive T-cell, the rate of activated T cell in the present compound group decreased, which shows that the present compound has an inhibitory effect for the activation of T cell. In addition, the present compound showed a higher inhibitory activity than each comparative drug, 5-aminosalicylic acid (5-ASA), rifaximin (RFX), ciprofloxacin (CPFX), or azathioprine, and in particular, 30 μM of the present compound showed a higher inhibitory activity than prednisolone in both of CD4-positive T-cell and CD8-positive T-cell.

Cytokine Production

IFN-γ and TNF-α which are produced with the activation of T cell were measured by ELISA, their inhibition ratios were calculated according to the following formula, and the results were shown in FIG. 7. The assay was carried out by two-way Dunnett, based on DMSO group as a control.

Inhibition ratio (%)=100−((“Each measured value”−“Average of measured values without stimulus/compound”)/(“Average of measured values without compound”−“Average of measured values without stimulus/compound”))×100

The cytokine production (IFN-γ, TNF-α) in the present compound group decreased, which suggests that the present compound has an inhibitory effect of cytokine production for T cell. In addition, the present compound showed a higher activity inhibiting the cytokine production than 5-aminosalicylic acid (5-ASA), rifaximin (RFX), or ciprofloxacin (CPFX).

Rate of Proliferated T Cell

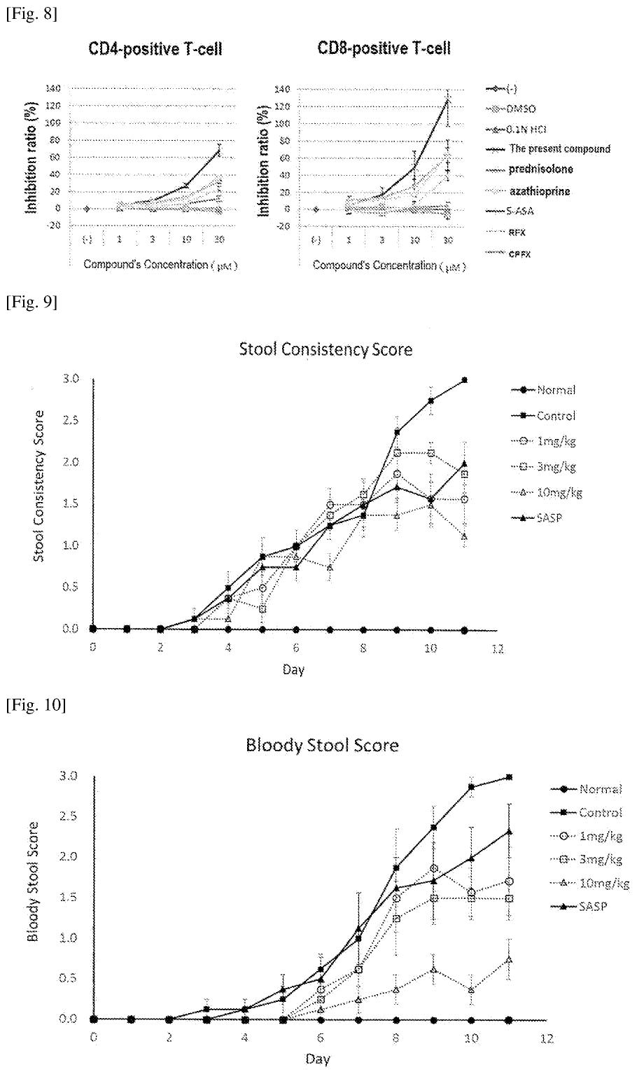

It is known that T cell is proliferated with the activation of cells. The ratios for inhibiting the proliferation with each test compound were shown in FIG. 8 using the fluorescence intensity of CFSE as an indicator. The inhibition ratio was calculated according to the following formula, and the assay was carried out by two-way Dunnett, based on DMSO group as a control.

Inhibition ratio (%)=100−((“Each value of MFI (CFSE)”−“Average of MFI (CFSE) values without stimulus/compound”)/(“Average of MFI (CFSE) values without compound”−“Average of MFI (CFSE) values without stimulus/compound”))×100

As for both of CD4-positive T-cell and CD8-positive T-cell, the results showed that the present compound group has an effect of cell growth inhibition. In addition, the effect of the present compound was more potent than that of all the comparative compounds.

### Example 5. Antibacterial Activity Against Fusobacterium Spp

There are plural reports about the relationship between intestinal bacteria and inflammatory bowel disease, and it is thought that Fusobacterium spp., one of the intestinal bacteria is a pathogenic bacteria causing inflammatory bowel disease. Thus, the antibacterial activity against 17 stains of Fusobacterium spp. was studied with the present compound, and ciprofloxacin (CPFX), rifaximin (RFX), and metronidazole (MTZ) which are comparative drugs.

(Method)

The antibacterial activity (minimum inhibitory concentration (MIC)) was assayed by agar plate dilution method which is based on Clinical and laboratory standards institute (CLSI). The medium used herein was Brucella medium with sheep blood. The bacterial cell was scraped out from the pre-culture medium, and prepared to the turbidity of 0.5 Mcfarland standard using Brucella Broth containing 5 μg/mL hemin and 1 μg/mL vitamin Kl. The bacterial suspension was inoculated onto Brucella agar with sheep blood which contains the present compound or any one of the comparative drugs, and incubated at 37° C. under an anaerobic condition.

(Result)

The MICs of the present compound, CPFX, RFX, and MTZ against the 17 stains of Fusobacterium spp. were 0.25-1.0, 2.0-8.0, 32->128, and 0.03-0.5 μg/mL, respectively. The present compound exhibited an antibacterial activity against Fusobacterium spp., whose antibacterial activity was the same or more than that of CPFX or RFX.

### Example 6. Antibacterial Activity Against Mycobacterium avium Subspecies Paratuberculosis

The chronic enterocolitis of ruminants which is caused by Mycobacterium avium subspecies paratuberculosis (MAP) is similar to Crohn's disease. If peripheral mononuclear cells derived from patients suffering from Crohn's disease are examined, it is found that 50-100% patients are infected with MAP, but healthy people are not so often infected. Hence, it is thought that MAP is a potential cause of Crohn's disease. Thus, the antibacterial activity of the present compound against MAP was evaluated. The used comparative drugs for the present compound were rifaximin (RFX), ciprofloxacin (CPFX), metronidazole (MTZ), clarithromycin (CAM), rifabutin (RBT), and clofazimine (CFZ).

(Method)

The antibacterial activity (minimum inhibitory concentration (MIC)) was assayed by broth microdilution method which is based on Clinical and laboratory standards institute (CLSI).

The 5 strains of MAP were inoculated onto Middlebrook 7H9 agar containing 10% dubos oleic albumin complex (OADC), Tween 80 (0.5 g/L), mycobactin J (2 mg/L), and 1.5% agar, and pre-cultured at 37° C. under an aerobic condition for 14 days. After the pre-culture, the bacterial cell was scraped out from the agar, and prepared to the turbidity of 0.5 Mcfarland standard using Middlebrook 7H9 broth containing 10% dubos oleic albumin complex (OADC), Tween 80 (0.5 g/L), and mycobactin J (2 mg/L)]. The 10-fold diluted solution of the bacterial suspension was inoculated onto Middlebrook 7H9 broth 96-well plate which contains the present compound or any one of the comparative drugs, and incubated at 37° C. under an aerobic condition for 14 days.

After the incubation, the bacterial growth in each well of the 96-well plate was visually checked. The minimum concentration at which the bacterial growth was not observed, compared with the result of the well containing neither present compound nor comparative drug was determined as MIC of the present compound or each comparative drug against each test bacterium.

(Result)

MICs of the present compound, CPFX, RFX, MTZ, CAM, RBT, CFZ, and RHB-104 against MAPS strain were 0.06-0.5, 0.25-0.5, 1-2, >128, 0.12-0.25, 0.25-1, 0.12-0.5, and 0.12-0.25 μg/mL, respectively. The MICs of the present compound against each test bacterium were in the same range as those of CPFX, CAM, RBT, RBT, and RHB-104. The MICs of the present compound were lower compared with those of RFX against each test bacterium. MTZ did not exhibit antibacterial activity against each test bacterium in the present test. In conclusion, the present compound exhibited the same or more antibacterial activity against MAP, compared with the comparative drugs.

### Example 7. Antibacterial Activity Against Intestinal Bacteria

The antibacterial activity of the present compound was examined against Bacteroides spp and Streptococcus spp, which are enterobacteria. The present compound exhibited strong antibacterial activities.

(Method)

The antibacterial activity (minimum inhibitory concentration (MIC)) was assayed by agar plate dilution method or broth microdilution method, which is based on Clinical and laboratory standards institute (CLSI). Specifically, the experiment was performed as follows. The compound of the present invention or the control substance was prepared by 2-fold serial dilution, respectively, and test strains were inoculated on the medium suitable for each test strain. After the inoculation followed by incubation at 37° C. under aerobic or anaerobic conditions, the minimum concentration at which colony formation was not observed in agar plate dilution method, or the minimum concentration without turbidity of the culture medium (the bacterial growth was not observed) in broth microdilution method was determined as MIC.

### Example 8. Effects for DSS-Induced Colitis Model

A colitis model induced by dextran sulfate sodium (DSS) is widely used as an experimental ulcerative colitis model having similarities of human ulcerative colitis regarding suppression of weight gain, colitis symptoms such as bloody stools and diarrhea, and injury formation in the large intestine. The effect of the present compound on the ulcerative colitis model was examined, and it was compared with salazosulfapyridine (SASP) clinically used as a therapeutic agent for ulcerative colitis.

(Method)

Symptoms of colitis were induced by allowing rats to freely drink 3% dextran sulfate sodium (DSS) solution for 10 days. Symptoms of feces in each group were observed for 11 days from the start day of 3% DSS solution freely drinking, and they are evaluated using the following scores as symptom of colitis. For the observation of stool symptoms, the degree of stool condition was scored as the score of stool firmness (Stool Consistency Score: 0, 1, 2, 3) and blood stool (Bloody Stool Score: 0, 1, 2, 3). The total of Stool Consistency Score and Bloody Stool Score was taken as the Total Stool Score. Each group was composed of 8 animals. Salazosulfapyridine (SASP), which is an already-existing drug for treating inflammatory bowel disease, the present compound or the vehicle were administrated for 10 days from the start date of 3% DSS freely drinking.

(Result)

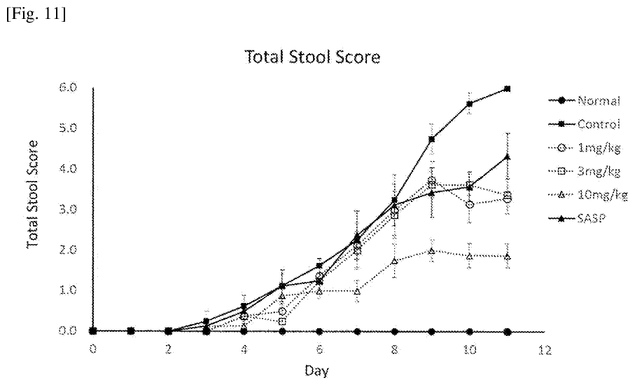

The present compound (1, 3, 10 mg/kg) or SASP (100 mg/kg) was orally administered twice a day from the start day of 3% DSS freely drinking, and fecal symptoms were observed daily. The results are shown in FIGS. 9, 10, and 11. In the vehicle control group, the stool score gradually worsened from the third day, reaching the maximum value on the 11th day, and 3 deaths were observed. In SASP group, suppression of stool score deterioration was observed from the 9th day, but 2 deaths were observed. On the 11th day, there were significant differences (P<0.05) between the vehicle control group and SASP group in stool firmness and total score, but blood stool score was not significant (Dunnett's test). In the present compound groups, the deterioration suppression effect on blood stool score from the 6th day and on stool firmness score from the 9th day were observed, and the remarkable deterioration suppression effect on blood stool score was observed at 10 mg/kg. One death was observed in the 1 mg/kg group, but no death in the 3, 10 mg/kg group. Significant differences were observed between the vehicle control group and the present compound groups in the stool firmness, bloody stool and total score on the 11th day (bloody stool score at 1 mg/kg P<0.05, other scores P<0.01) (Dunnett's Test).

According to these results, the present compound decreased the number of death compared with the vehicle control group and SASP group, and significantly inhibited the deterioration of stool symptoms, especially remarkably inhibited the deterioration of bloody stool.

1. A medicament comprising a quinolone compound of formula (I): 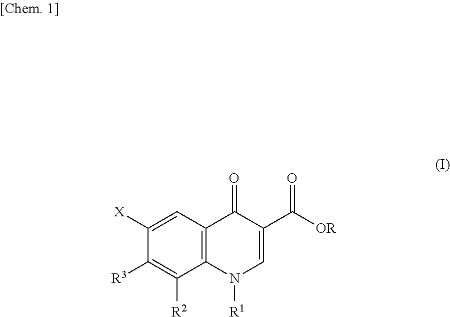
or a pharmaceutically acceptable salt thereof,
wherein
X is hydrogen atom or fluorine atom;
R is hydrogen atom or alkyl;
R¹ is (1) cyclopropyl which may be optionally substituted with 1 to 3 the same or different halogen atoms, or (2) phenyl which may be optionally substituted with 1 to 3 the same or different halogen atoms;
R² is hydrogen atom; alkyl which may be optionally substituted with 1 or 2 substituents selected independently from the group consisting of halogen atom and hydroxy; alkoxy; haloalkoxy; halogen atom; cyano; cyclopropyl; nitro; amino; formyl; alkenyl; or alkynyl; or
R¹ and R² are taken together to form 5- or 6-membered ring which may be optionally substituted with alkyl;
R³ is
(1) a fused heterocyclyl group of the formula: 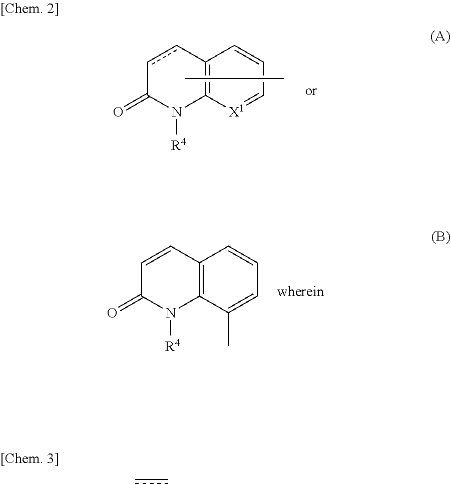
represents single bond or double bond,
X¹ is C(R⁵) or N,
R⁴ is hydrogen atom or alkyl,
R⁵ is
(a) hydrogen atom,
(b) halogen atom,
(c) cyano,
(d) nitro,
(e) hydroxy,
(f) alkyl which may be optionally substituted with 1 to 3 the same or different halogen atoms,
(g) alkenyl or alkynyl,
(h) aryl, or
(i) alkoxy which may be optionally substituted with 1 to 3 the same or different halogen atoms, when X¹ is C(R⁵), R⁴ and R⁵ may be taken together to form 5- or 6-membered ring which may be optionally substituted with oxo,
said fused heterocyclyl group may be optionally substituted with 1 or 2 substituents selected independently from the group consisting of halogen atom, cyano, nitro, hydroxy, and alkyl,
(2) a group of the formula: 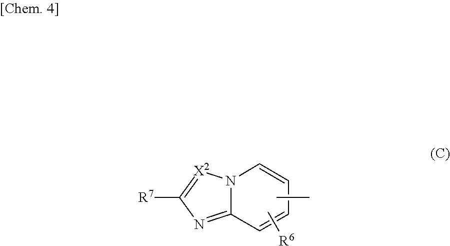
wherein
X² is C(R⁸) or N, and
R⁶, R⁷, and R⁸ are each independently,
(a) hydrogen atom,
(b) halogen atom,
(c) cyano,
(d) nitro,
(e) amino,
(f) alkyl which may be optionally substituted with 1 to 3 substituents selected independently from the group consisting of halogen atoms, alkoxy, and amino,
(g) alkenyl,
(h) alkynyl,
(i) aryl,
(j) formyl or CH═N—OH,
(k) carboxy,
(l) carbamoyl,
(m) 5- to 10-membered aromatic heterocyclyl group which may be optionally substituted with alkyl, or
(n) alkenyloxy,
(3) a group of the formula: 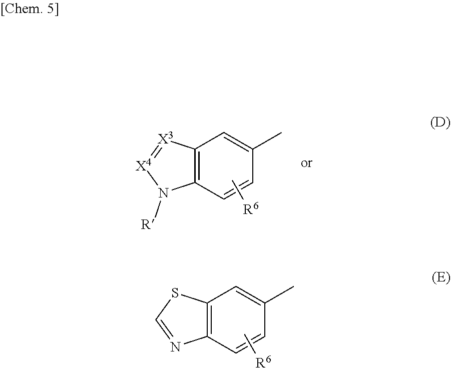
wherein
X³ and X⁴ are N, or
X³ is N, and X⁴ is CR″, wherein R″ is hydrogen atom; amino; hydroxy; alkyl which may be optionally substituted with 1 to 3 substituents selected independently from the group consisting of alkoxy and dimethylamino; or mercapto, or
X³ is CH and X⁴ is N,
R′ is hydrogen atom, or alkyl which may be optionally substituted with 1 to 3 substituents selected from the group consisting of substituted hydroxyl and amino, and
R⁶ is as defined above,
(4) a group of the formula: 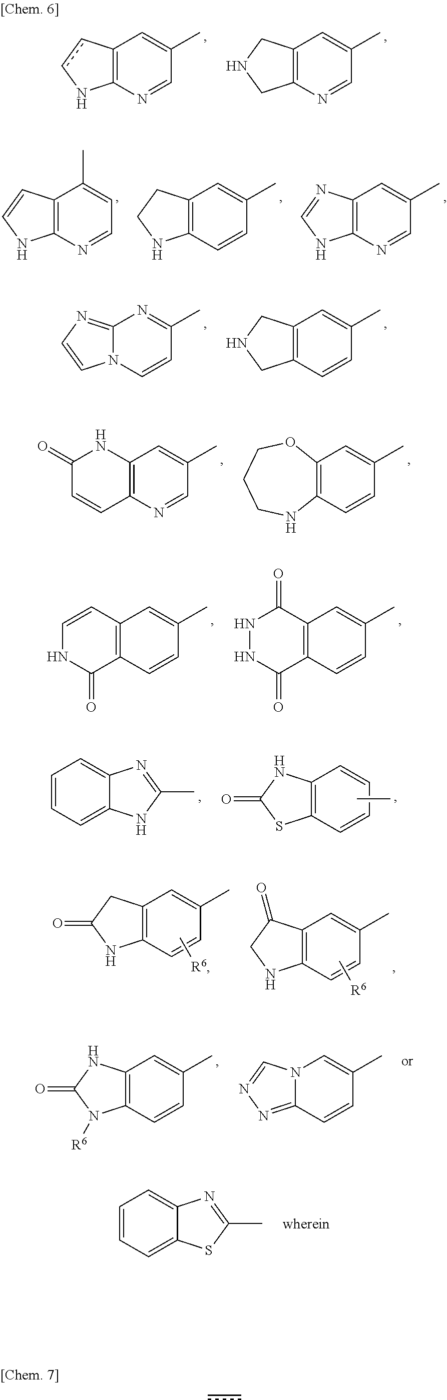
represents single bond or double bond, and R⁶ is as defined above,
(5) 3-pyridyl which may be optionally substituted with 1 to 2 substituents selected independently from the group consisting of the following (a)-(q):
(a) halogen atom,
(b) cyano,
(c) nitro,
(d) hydroxy,
(e) amino,
(f) alkyl which may be optionally substituted with 1 to 3 substituents selected independently from the group consisting of halogen atom, alkylamino, dialkylamino, and hydroxy,
(g) alkenyl or alkynyl,
(h) aryl,
(i) cycloalkyl,
(j) alkoxy,
(k) alkylamino,
(l) dialkylamino,
(m) phenylamino which may be optionally substituted with 1 to 3 the same or different halogen atoms,
(n) cyclic amino group which may be optionally substituted with alkoxycarbonyl,
(o) formyl,
(p) carbamoyl which may be optionally substituted with alkyl optionally-substituted with hydroxy, and
(q) 5- to 10-membered aromatic heterocyclyl group which may be optionally substituted with alkyl,
(6) 4-pyridyl which may be optionally substituted with halogen atom,
(7) 5-pyrimidinyl which may be optionally substituted with 1 or 2 substituents selected independently from the group consisting of amino, alkylamino, dialkylamino, and carboxy,
(8) 2-indolyl, 3-indolyl, 5-indolyl, 6-indolyl, benzofuranyl, benzothiophenyl, benzoxazolyl, or benzothiazolyl, each of which may be optionally substituted with 1 or 2 substituents selected independently from the consisting of the following (a)-(j):
(a) halogen atom,
(b) cyano,
(c) nitro,
(d) hydroxy,
(e) alkyl which may be optionally substituted with 1 to 3 substituents selected independently from the group consisting of amino, alkoxycarbonylamino, alkylamino, and dialkylamino,
(f) alkoxy,
(g) formyl,
(h) carboxy, and
(j) amino which may be optionally substituted with 1 or 2 substituents selected independently from the group consisting of the following (i)-(x):
(i) alkoxycarbonyl,
(ii) alkylcarbonyl which may be optionally substituted with a substituent selected from the group consisting of the following (A)-(E):
(A) cycloalkyloxy which may be optionally substituted with 1 to 3 the same or different alkyl,
(B) alkylamino,
(C) dialkylamino,
(D) cyclic amino group which may be optionally substituted with alkoxycarbonyl, and
(E) halogen atom,
(iii) phenylcarbonyl which may be optionally substituted with 1 to 3 substituents selected independently from the group consisting of alkyl and alkoxy,
(iv) cycloalkylcarbonyl,
(v) 5- to 10-membered aromatic heterocyclylcarbonyl group which may be optionally substituted with alkyl optionally-substituted with 1 to 3 the same or different halogen atoms,
(vi) benzylcarbonyl which may be substituted with 1 to 3 substituents selected independently from the group consisting of halogen atom and alkoxy,
(vii) arylsulfonyl which may be optionally substituted with alkoxy,
(viii) cycloalkylalkylsulfonyl which may be optionally substituted with 1 to 3 substituents selected independently from the group consisting of alkyl and oxo,
(ix) 5- to 10-membered aromatic heterocyclylsulfonyl group which may be optionally substituted with 1 to 3 the same or different alkyl, and
(x) —C(═N—CN)—SR⁹ wherein R⁹ is alkyl,
(9) a group of the formula: 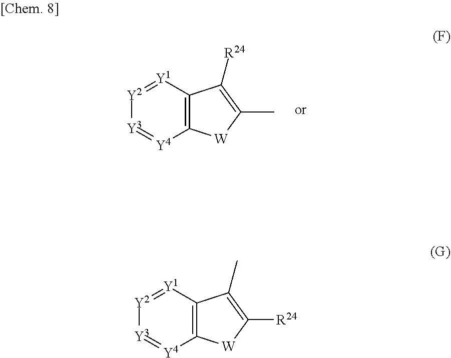
wherein
one of Y¹, Y², Y³, and Y⁴ is N or N⁺(—O⁻), and the remaining three are either different one of
C(R²⁵), C(R²⁶), and C(R²⁷),
W is O, S, or N(R²³),
R²³ is hydrogen atom or alkyl, and
R²⁴, R²⁵, R²⁶, and R²⁷ are each independently
(a) hydrogen atom,
(b) cyano, or
(c) nitro,
(10) a group of the formula: 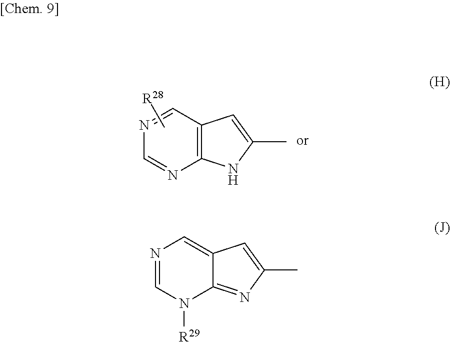
wherein
R²⁸ is hydrogen atom or hydroxy, and
R²⁹ is hydrogen atom or alkyl,
(11) a group of the formula: 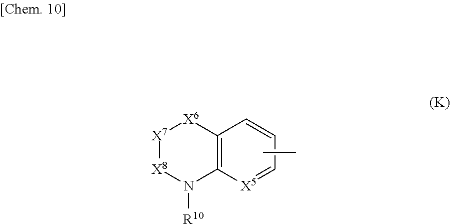
wherein
X⁵ is C(R¹¹) or N,
X⁶ is CH₂, C(═O), O, S, SO₂, or N(R¹²),
X⁷ is CH(R¹³), C(═O), or N(R¹⁴),
X⁸ is CH(R¹⁵) or C(═O),
R¹⁰, R¹², and R¹⁴ are each independently
(a) hydrogen atom or
(b) alkyl,
R¹¹, R¹³, and R¹⁵ are each independently
(a) hydrogen atom,
(b) halogen atom,
(c) cyano,
(d) nitro,
(e) amino,
(f) alkylamino,
(g) dialkylamino,
(h) alkyl which may be optionally substituted with hydroxy, or
(i) alkenyl,
when X⁵ is C(R¹¹), R¹⁰ and R¹¹ may be taken together to form 5- or 6-membered ring which may be optionally substituted with alkyl or oxo, and
when X⁶ is N(R¹²) and X′ is CH(R¹³), R¹² and R¹³ may be taken together to form 5- or 6-membered ring,
(12) a group of the formula: 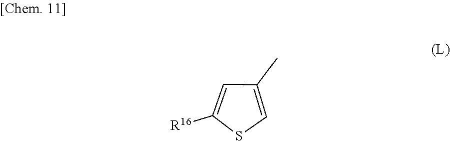
wherein R¹⁶ is
(a) hydrogen atom,
(b) alkyl which may be optionally substituted with 1 to 3 substituents selected from the group consisting of cyano, alkylamino, and dialkylamino,
(c) alkenyl which may be optionally substituted with carboxy,
(d) formyl,
(e) carboxy,
(f) carbamoyl,
(g) —C(R¹⁷)═N—OH wherein R¹⁷ is hydrogen atom, cyano, or hydroxy,
(h) 5- to 10-membered aromatic heterocyclyl group which may be optionally substituted with alkyl, alkoxycarbonyl, carboxy, or phenyl, or
(i) cyano,
(13) a group of the formula: 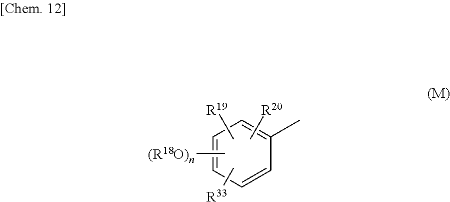
wherein
R¹⁸ is hydrogen atom, or alkyl which may be optionally substituted with 1 to 3 substituents selected independently from the group consisting of halogen atoms and phenyl,
n is 0 or 1,
R⁹, R²⁰, and R³³ are each independently,
(a) hydrogen atom,
(b) halogen atom,
(c) cyano,
(d) alkyl which may be optionally substituted with 1 to 3 substituents selected independently from the group consisting of the following (i)-(vii):
(i) halogen atom,
(ii) cyano,
(iii) hydroxy,
(iv) amino,
(v) alkylamino,
(vi) dialkylamino, and
(vii) cyclic amino group which may be optionally substituted with alkyl,
(e) alkoxy,
(f) amino which may be optionally substituted with 1 or 2 substituents selected independently from the following (i)-(v):
(i) alkylcarbonyl which may be optionally substituted with cyclic amino group,
(ii) alkylsulfonyl,
(iii) carbamoyl,
(iv) alkyl, cycloalkyl, or cycloalkylalkyl, and
(v) 5- to 10-membered saturated heterocyclyl,
(g) carboxy,
(h) alkoxycarbonyl,
(i) carbamoyl which may be optionally substituted with alkyl optionally-substituted with amino, alkylamino, dialkylamino, or alkoxycarbonylamino,
(j) formyl,
(k) 5- to 10-membered aromatic heterocyclyl group which may be optionally substituted with alkyl,
(l) —CH═N—OR²¹ wherein R²¹ is hydrogen atom, or alkyl which may be substituted with alkylamino or dialkylamino,
(m) nitro,
(n) 5- to 10-membered saturated heterocyclyl which may be optionally substituted with amino,
(o) phenyl, or
(p) —NHC(SMe)=CHCN,
(14) a group of the formula: 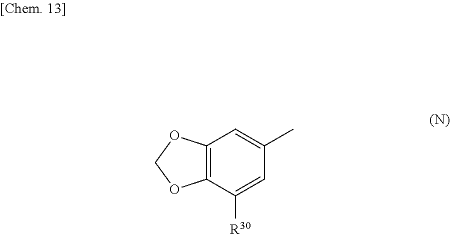
wherein
R³⁰ is
(a) hydrogen atom,
(b) halogen atom,
(c) cyano,
(d) alkyl which may be optionally substituted with 1 to 3 substituents selected independently from the group consisting of halogen atom and hydroxy,
(e) alkenyl,
(f) alkynyl,
(g) alkoxy,
(h) formyl,
(i) —CH═N—OH, or
(j) carbamoyl,
(15) naphthyl or isochromenyl,
(16) quinolyl or isoquinolyl, or oxide form thereof,
(17) a group of the formula: 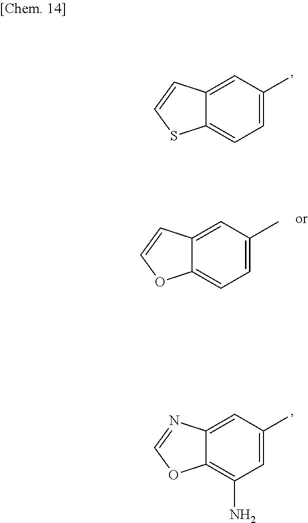
(18) a group of the formula: 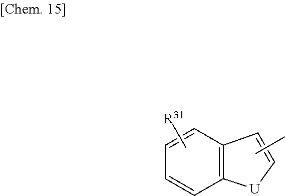
wherein
U is O or S, and
R³¹ is
(a) hydrogen atom,
(b) halogen atom,
(c) alkyl which may be optionally substituted with 1 to 3 the same or different halogen atoms,
(d) carboxy,
(e) nitro,
(f) cyano, or
(g) amino,
(19) a group of the formula: 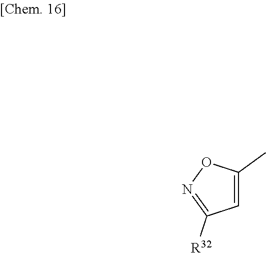
wherein
R³² is
(a) halogen atom,
(b) phenyl, or
(c) a group of the formula: 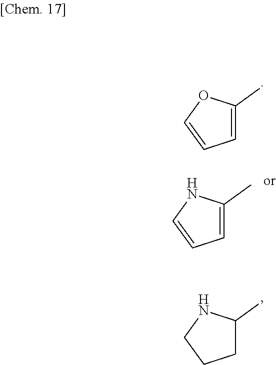
(20) a group of the formula: 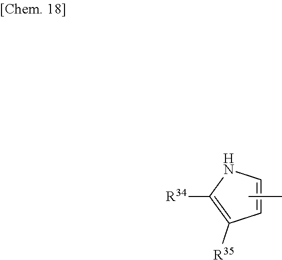
wherein
R³⁴ and R³⁵ are each independently
(a) hydrogen atom, or
(b) aminoalkyl, or
R³⁴ and R³⁵ are taken together to form 6-membered ring which may be optionally substituted with amino or oxo,
(21) a group of the formula: 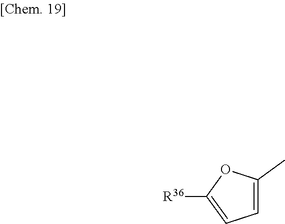
wherein R³⁶ is
(a) hydrogen atom,
(b) halogen atom,
(c) nitro, or
(d) thienyl, or
(22) a group of the formula: 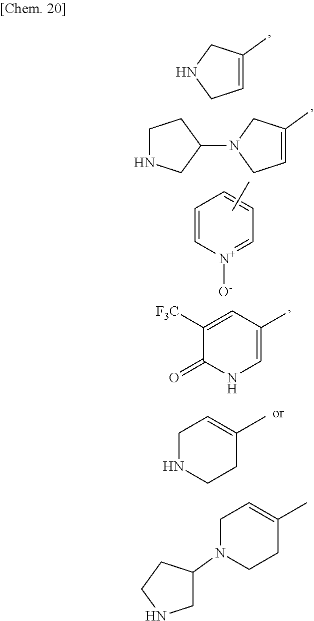
for treating and/or preventing a disease connected with the change of enteric bacteria, or a disease involving inflammation.

2. The medicament of claim 1 wherein the disease connected with the change of enteric bacteria, or the disease involving inflammation is inflammatory bowel disease.

3. The medicament of claim 2 wherein the inflammatory bowel disease is Crohn's disease or ulcerative colitis.

4. The medicament of claim 2 wherein the inflammatory bowel disease is Crohn's disease.

5. The medicament of claim 1, which is an oral preparation.

6. The medicament of claim 1, wherein the daily dose is 0.5 mg-6000 mg.

7. A method for treating and/or preventing inflammatory bowel disease, comprising administering a therapeutically effective amount of the quinolone compound defined in claim 1 or a pharmaceutically acceptable salt thereof to a patient in need thereof.

8. Use of the quinolone compound defined in claim 1 or a pharmaceutically acceptable salt thereof in the manufacture of a medicament for treating and/or preventing inflammatory bowel disease.

9. The quinolone compound defined in claim 1 or a pharmaceutically acceptable salt thereof for use in treating and/or preventing inflammatory bowel disease.

10. A medicament comprising the quinolone compound defined in claim 1 or a pharmaceutically acceptable salt thereof, which has an antibacterial activity against bacteria involved in inflammatory bowel disease, an inhibitory action for inflammatory cytokine production, and an inhibitory action for activation of T cell.

11. The medicament of claim 1, which is an oral preparation, wherein the medicament has a bioavailability of 5% or less.

12. The method of claim 7 wherein the daily dose is 0.5 mg-6000 mg.

13. The method of claim 7 wherein the inflammatory bowel disease is Crohn's disease or ulcerative colitis.

14. The method of claim 7 wherein the inflammatory bowel disease is Crohn's disease.
